(19) United States
(12) Patent Application Publication
(10) Pub. No.: US 2023/0417773 A1
(43) Pub. Date: Dec. 28, 2023

(54) DIAGNOSTIC ASSAYS EMPLOYING NEURON-DERIVED EXOSOMES

(71) Applicant: NEURODEX, INC., Natick, MA (US)

(72) Inventor: Erez Eitan, Arlington, MA (US)

(21) Appl. No.: 18/245,140

(22) PCT Filed: Sep. 14, 2021
(86) PCT No.: PCT/IB2021/058351

§ 371 Date: Mar. 13, 2023

Related U.S. Application Data
(60) Provisional application No. 63/078,363, filed on Sep. 15, 2020.

(51) Int. Cl. G01N 33/68; G01N 33/50; G01N 33/543

(52) U.S. Cl. CPC G01N 33/6896; G01N 2800/2821; G01N 33/54326; G01N 33/5058

(57) ABSTRACT

The present invention relates to minimally invasive, biomarker-based diagnostics for neurodegenerative diseases, and to compositions and methods for isolating and analyzing specific populations of extracellular vesicles (EV). In particular, embodiments of the invention relate to methods and systems for isolating, identifying or capturing neuron-derived EV, for analyzing biological samples, and for diagnosing, assessing and predicting the development of neurological and neurodegenerative conditions.

### FIELD OF THE INVENTION

The present invention relates to minimally invasive, biomarker-based diagnostics for neurodegenerative diseases, and to compositions and methods for isolating specific exosomes and analyzing their contents.

### BACKGROUND OF THE INVENTION

There are very few approved treatments for neurodegenerative diseases, and the development of disease-modifying therapies for these diseases is considered one of the most challenging tasks in the medical field. Some of the challenges involved in this task are due to insufficiency of the diagnostic toolbox available. For example, the phytological accumulation of misfolded proteins in Alzheimer's Disease (AD) and Parkinson's Disease (PD) begins many years before onset of clinical symptoms and diagnosis (Petersen, Neurology 91(9): 395-402, 2018).

Therefore, when diagnosed at a later, symptomatic stage, the neurons are already fully deteriorated, or severely damaged, rendering most treatments ineffective. Another shortcoming is evident in the fact that postmortem analysis demonstrates that 20%-30% of AD patients are misdiagnosed, and suffer from other types of dementia.

Current diagnosis of such neurological diseases relies on clinical testing, brain-imaging and cerebrospinal fluid (CSF) biomarkers. While advancements in brain-imaging and CSF biomarkers were incorporated into clinical trials, these are expensive, invasive and/or require exposure to radiation, and are therefore not appropriate for population screening or longitudinal monitoring. Inexpensive and minimally invasive blood-based diagnostic tests could address these needs. In addition, population screening may help identifying AD and other neurological diseases earlier and/or in a more accurate manner. The development of blood tests can also allow routine and longitudinal monitoring of disease progression and response to treatments. Further, in many cases the diagnosis process is long and cumbersome. For example, amyotrophic lateral sclerosis (ALS) diagnosis takes about 14 months on average. The process until the diagnosis is confirmed is complex, involves evaluation by multiple physicians, and involves uncertainties for physicians and patients alike.

Extracellular vesicles (EV) are membranous particles shed by all cells and found in all biofluids; they include exosomes (30 nm to 150 nm in diameter) originating from endosomes/multivesicular bodies, and microvesicles (150 nm to 1000 nm) produced through budding of the plasma membrane. EV contain protein and RNA of their cell of origin and it has been shown that they can contain a great multitude of biomarkers, which may be potentially relevant for various diagnostic purposes (Keerthikumar et al., J Mol Biol 428(4): 688-692, 2016). In addition, EV may contain many surface proteins, some of which unique to cells of a certain origin. One challenge in employing the use of EV in diagnostics is that most cellular markers that are currently in clinical use have been discovered using histology or fluorescence-activated cell sorting (FACS) assays, where the levels of the protein markers are compared to those of control cells in their environment. Discovering a marker selective for EV derived from specific origins (e.g. cell types or tissue) requires that this marker is either unique to these EV and/or cell types from which they are derived, or is found on these EV at higher levels than on EV derived from other cell types.

Previous work has suggested the use of L1 Cell Adhesion Molecule (L1CAM)-specific antibodies to select neuron-derived exosomes. However, while L1CAM is highly expressed in neurons, it is also expressed in lymph nodes, kidneys and some activated lymphocytes, thereby impairing its use as an isolation target in potential diagnostic assays. A recent publication to the inventor and coworkers relates to association of extracellular vesicle biomarkers with AD in the Baltimore Longitudinal Study of Aging (Kapogiannis et al., JAMA Neurol., 76(11):1340-1351, 2019). The study shows that L1CAM-based exosome isolation can identify stage 1 AD (in 131 pre-symptomatic AD patients) upon longitudinal measurement of various biomarkers at different time points over several years, using a small-scale laboratory assay of EV isolation. Other studies have examined various approaches, including those involving enrichment of neuronal-derived exosomes, in an attempt to develop markers for other neurological disorders such as PD, Frontotemporal dementia (FTD), traumatic brain injury (TBI) and HIV-related dementia (Osier et al. Mol Neurobiol. 2018; 55(12):9280-9293.

Thus, hitherto reported methods involving e.g. selection utilizing L1CAM-specific antibodies isolate both neuronal exosomes and various non-neuronal exosomes, therefore offering only slight enrichment of the desired exosome population and an unsatisfactory accuracy.

U.S. Pat. No. 9,958,460 relates to biomarkers and diagnostic and prognostic methods for AD and other neurodegenerative disorders. U.S. '460 also provides compositions for detecting the biomarker as well as compositions and methods useful for treating AD and other neurodegenerative disorders. In particular, the publication discloses the detection of certain biomarkers in vesicles obtained from a biological sample. WO2017193115 relates to synaptic protein biomarkers for differential diagnosis of AD and other neurodegenerative disorders. In particular, the publication discloses a method of analyzing a sample from a subject comprising the steps of: (i) obtaining a biological sample comprising vesicles from the subject, (ii) measuring the level of one or more biomarkers in the biological sample, and (iii) comparing the level of the one or more biomarkers in the biological sample to a control level of the one or more biomarkers in a control biological sample, wherein at least one or more biomarkers are selected from the group consisting of synaptophysin, synaptopodin, synaptotagmin, neurogranin, and human growth associated protein 43 (GAP43). Other publications involving the isolation and/or analysis of vesicles from biological samples include e.g. US20190219578, US20180340945, US20190361037, US20180080945, US20190137517 and WO2016172598.

A recent publication to the inventor and coworkers, published after the priority date of the present application, relates to neuronal and astrocytic extracellular vesicle biomarkers in blood reflect brain pathology in mouse models of AD (Delgado-Peraza et al., 2021, Cells 10, 993). There remains an unmet medical need for improved diagnostic assays for neurodegenerative disorders. The development of minimally invasive, biomarker-based diagnostic platform, enabling early and accurate detection of various neurological disorders such as AD and dementia, as well as means for differential diagnosis, prognosis and monitoring of such disorders, would be highly advantageous. In addition, methods and means providing for specific isolation of neuron-derived exosomes and analysis of their payload, would afford considerable benefits in experimental and clinical settings alike.

### SUMMARY OF THE INVENTION

The present invention relates to minimally invasive, biomarker-based diagnostics for neurodegenerative diseases, and to compositions and methods for isolating and analyzing specific populations of extracellular vesicles (EV). In particular, embodiments of the invention relate to methods and systems for isolating, identifying or capturing neuron-derived EV, for analyzing biological samples, and for diagnosing, assessing and predicting the development of neurological and neurodegenerative conditions.

The invention is based, in part, on the surprising identification of improved assays and methods for isolating and analyzing neuron-derived extracellular vesicles (NDE). The assays and methods of the invention advantageously employ the use of synergistic combinations of substance-bound affinity molecules directed to synaptic proteins, thereby targeting multiple synaptic proteins on the surface of NDE. Further advantageous assays and methods of the invention are based on improved protocols comprising synthetic or recombinantly-produced control particles. The invention is further based in part on the unexpected discovery of highly accurate diagnostic assays for various neurological diseases, enabling early detection and characterization of the disease or pathology by analyzing peripheral blood samples. In particular, disclosed herein are a highly accurate diagnostic classifier for Mild Cognitive Impairment (MCI) based on a combination of four protein markers, and assays for differential diagnosis of frontotemporal dementia (FTD) pathophysiology. In addition, remarkably accurate diagnostic classifiers for amyotrophic lateral sclerosis (ALS; based on a combination of three biomarkers), and for Alzheimer's disease (AD; combining the levels of four protein markers), were also developed using the systems and methods of the invention.

In one aspect, there is provided a method for isolating neuron-derived EV, comprising:
a. providing an isolation system comprising substance-bound affinity molecules capable of binding to target molecules on the surface of the EV, wherein at least one of the target molecules is a synaptic protein,
b. providing a control sample containing a predetermined amount of particles that display one or more of the target molecules,
c. determining the accuracy of the system, by:
   i. contacting said system with the control sample, under conditions enabling specific binding of said affinity molecules to their corresponding target molecules,
   ii. quantifying the amount of particles bound by said affinity molecules, and
   iii. determining that the amount of bound particles is above a predetermined threshold,
d. providing an EV-containing biofluid sample,
e. contacting the biofluid sample with said system, under the conditions enabling specific binding of said affinity molecules to their corresponding target molecules, and
f. isolating the EV bound to said affinity molecules.

In one embodiment, said synaptic protein is selected from the group consisting of neuroligin-3 (NLGN3), growth-associated protein 43 (GAP43), Synaptotagmin-1 (SYT1), and Glutamate receptor 1 (GluR1, GRIA1). In another embodiment, said target molecules comprise NLGN3 and GAP43. In another embodiment, said target molecules are NLGN3 and GAP43. In yet another embodiment, said target molecules further comprise L1 Cell Adhesion Molecule (L1CAM) and/or Rab3a. In another embodiment, said target molecules are NLGN3, GAP43 and L1CAM.

In another embodiment, the particles of the control sample are labeled by a marker. In another embodiment said particles are beads (including, but not limited to, microparticle or nanoparticle beads). In a particular embodiment said particles are fluorescently labeled beads, and the control sample is combined with the biofluid sample prior to contacting said biofluid sample with said affinity molecules. In yet another embodiment, the particles are control EV obtained from engineered cells. In another embodiment, the control sample comprises control EV comprising positive control EV engineered to express the one or more of the target molecules exogenously, and optionally further comprises negative control EV that do not express said target molecules. In a particular embodiment, said control sample comprises: a) negative control EV, obtained from non-neuronal cells that contain a first fluorescent marker, and b) positive control EV, obtained from equivalent non-neuronal cells engineered to express said one or more of the target molecules, and containing a second, distinct fluorescent marker. In another embodiment the predetermined threshold corresponds to recovery of at least 44% of the predetermined amount of particles provided in the control sample (i.e. target-displaying particles such as positive control EV or beads).

In another embodiment, the isolated EV are characterized by a ratio of neuron-specific marker levels to non-neuron-specific marker levels of at least 20-fold. In another embodiment, the substance (to which the affinity molecules are bound) is a plurality of magnetic beads. In another embodiment the affinity molecules are antibodies or comprise an antigen-binding portion thereof. In another embodiment the biofluid sample is selected from the group consisting of blood, plasma, and serum. In another embodiment said subject is human.

In another embodiment, the method further comprises determining the levels of one or more biomarkers in the isolated EV. In another embodiment, the one or more biomarkers are selected from the group consisting of protein biomarkers, nucleic acid biomarkers, lipid biomarkers, metabolite biomarkers, and combinations thereof. In another embodiment, the one or more biomarkers are selected from the group consisting of Microtubule Associated Protein Tau (Tau), phosphorylated Tau (p-Tau), Amyloid-beta 42 (Aβ42), and NLGN, TDP43, clusterin (CLU), SYP, BIM, NEFL, ENO2, NRGN, and GPR26 gene products, and combinations thereof. In various other embodiments, the one or more biomarkers are selected from the group consisting of the one or more biomarkers are selected from the group consisting of Tau, p-Tau, Aβ42, and NLGN, TDP43, CLU, SYP, BIM, NEFL, ENO2, NRGN, Cathepsin D, LC3, SYT and GPR26 gene products, and combinations thereof. In yet other embodiments, the one or more biomarkers are selected from the group consisting of: LC3, Cathepsin D, NRF2, Aβ42, p-Tau, PSD95, proBDNF, COX2, EIF2C2 and NF-κB. Each possibility represents a separate embodiment of the invention.

In another embodiment, the sample is obtained from a subject suspected of having MCI, or of being predisposed to developing AD. In another embodiment, the biomarkers are Tau (also referred to herein as “total-Tau”), p-Tau, Aβ42, and Neuroligin. In another embodiment, the sample is obtained from a subject suspected of having MCI, and/or of being predisposed to developing AD, and the biomarkers are Tau, p-Tau, Aβ42, and Neuroligin. In another embodiment, the method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with MCI. In another embodiment, the method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is predisposed to developing AD (e.g. to develop symptomatic AD within 3-5 years of obtaining the biofluid sample).

In another embodiment the sample is obtained from a subject diagnosed with, or suspected of having, FTD, and the biomarkers are TDP43 and p-Tau. In another embodiment said method further comprises characterizing the FTD pathology in said subject based on the levels of TDP43 and p-Tau determined in the isolated EV.

In another embodiment, said biomarkers are Aβ42, p-Tau, PSD95 and proBDNF, and the sample is obtained from a subject suspected of having AD. In another embodiment, said method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with AD.

In another embodiment, said biomarkers are LC3, TDP43, and NRF2, and the sample is obtained from a subject suspected of having ALS. In another embodiment, the method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with ALS.

In another aspect, there is provided a method of identifying or capturing neuron-derived EV, comprising:
a. providing an EV-containing biofluid sample,
b. contacting the sample with affinity molecules capable of binding to target molecules on the surface of the EV, under conditions enabling specific binding of the affinity molecules to their corresponding target molecules, wherein said target molecules comprise NLGN3 and GAP43, and
c. identifying or capturing the EV bound to the affinity molecules.

In another embodiment said target molecules are NLGN3 and GAP43. In another embodiment said target molecules further comprise L1CAM. In another embodiment said target molecules are NLGN3, GAP43 and L1CAM. In another embodiment said affinity molecules are substance-bound. In another embodiment the substance is a plurality of magnetic beads.

In another embodiment, the method further comprises:
i. providing a control sample containing a predetermined amount of particles that display at least one of the target molecules,
ii. contacting the control sample with the affinity molecules, under the conditions enabling specific binding to their corresponding target molecules,
iii. quantifying the amount of particles bound by said affinity molecules, and
iv. determining that the amount of bound particles is above a predetermined threshold.

In another embodiment, said particles are fluorescently labeled beads, and the control sample is combined with the biofluid sample prior to contacting said biofluid sample with said affinity molecules. In another embodiment said control sample comprises positive control EV engineered to express the one or more of the target molecules exogenously, and optionally further comprises negative control EV that do not express said target molecules. In another embodiment said control sample comprises negative control EV obtained from non-neuronal cells that contain a first fluorescent marker, and positive control EV obtained from equivalent non-neuronal cells engineered to express the one or more of the target molecules, and containing a second, distinct fluorescent marker. In another embodiment, the predetermined threshold corresponds to recovery of at least 44% of the predetermined amount of particles (that display the one or more of said target molecules) provided in the control sample.

In another embodiment the isolated EV are characterized by a ratio of neuron-specific marker levels to non-neuron-specific marker levels of at least 20-fold. In another embodiment the substance is a plurality of magnetic beads. In another embodiment the affinity molecules are antibodies or comprise an antigen-binding portion thereof. In another embodiment the biofluid sample is selected from the group consisting of blood, plasma and serum. In another embodiment said subject is human.

In another embodiment the method further comprises determining the levels of one or more biomarkers in the EV bound to the affinity molecules. In another embodiment, the one or more biomarkers are selected from the group consisting of protein biomarkers, nucleic acid biomarkers, lipid biomarkers, metabolite biomarkers, and combinations thereof. In another embodiment, the one or more biomarkers are selected from the group consisting of Tau, phosphorylated Tau (p-Tau), Aβ42, and NLGN, TDP43, clusterin, SYP, BIM, NEFL, ENO2, NRGN, and GPR26 gene products, and combinations thereof.

In another embodiment, the sample is obtained from a subject suspected of having MCI, or of being predisposed to developing AD. In another embodiment, the biomarkers are total-Tau (Tau), p-Tau, Aβ42, and Neuroligin. In another embodiment, the sample is obtained from a subject suspected of having MCI, or of being predisposed to developing AD, and the biomarkers are Tau, p-Tau, Aβ42, and Neuroligin. In another embodiment, the method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with MCI. In another embodiment the method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is predisposed to developing AD.

In another embodiment the sample is obtained from a subject diagnosed with, or suspected of having, FTD, and the biomarkers are TDP43 and p-Tau. In another embodiment said method further comprises characterizing the FTD pathology in said subject based on the levels of TDP43 and p-Tau determined in the isolated EV.

In yet other embodiments, the one or more biomarkers are selected from the group consisting of: LC3, Cathepsin D, NRF2, Aβ42, p-Tau, PSD95, proBDNF, COX2, EIF2C2 and NF-κB. In another embodiment, said biomarkers are Aβ42, p-Tau, PSD95 and proBDNF, and the sample is obtained from a subject suspected of having AD. In another embodiment, said method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with AD.

In another embodiment, said biomarkers are LC3, TDP43, and NRF2, and the sample is obtained from a subject suspected of having ALS. In another embodiment, the method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with ALS.

In another aspect, there is provided a method for analyzing a biofluid sample, comprising determining the levels of biomarkers in neuron-derived EV of the sample, wherein
a. the sample is obtained from a subject suspected of having MCI, or of being predisposed to developing AD, and the biomarkers are Tau, p-Tau, Aβ42, and Neuroligin; or
b. the sample is obtained from a subject diagnosed with, or suspected of having, FTD, and the biomarkers are TDP43 and p-Tau.

In another aspect, there is provided a method for analyzing a biofluid sample, comprising determining the levels of biomarkers in neuron-derived EV of the sample, wherein
a. the sample is obtained from a subject suspected of having MCI, or of being predisposed to developing AD, and the biomarkers are total-Tau (Tau), p-Tau, Aβ42, and Neuroligin (NLGN); or
b. the sample is obtained from a subject diagnosed with, or suspected of having, FTD, and the biomarkers are TDP43 and p-Tau; or
c. the sample is obtained from a subject suspected of having AD, and the biomarkers are Aβ42, p-Tau, PSD95 and proBDNF; or
d. the sample is obtained from a subject suspected of having ALS, and the biomarkers are LC3, TDP43, and NRF2.

In another embodiment, the method further comprises comparing the levels of the biomarkers in the EV to their respective levels corresponding to a control biofluid sample. In another embodiment, the method further comprises, prior to determining the levels of the biomarkers in the neuron-derived EV, a step of isolating said EV from the sample, by contacting said sample with substance-bound affinity molecules capable of binding to target molecules on the surface of said EV, wherein at least one target molecule is a synaptic protein, under conditions enabling specific binding of the affinity molecules to their corresponding target molecules, and isolating the EV bound to said affinity molecules. In another embodiment, said affinity molecules are directed to NLGN3 and GAP43 and the substance is a plurality of magnetic beads.

In another embodiment the method further comprises:
i. providing a control sample containing a predetermined amount of particles that display one or more of the target molecules,
ii. contacting the control sample with the affinity molecules, under the same conditions enabling specific binding to their corresponding target molecules,
iii. quantifying the amount of particles bound by said affinity molecules, and
iv. determining that the amount of bound particles is over a predetermined threshold.

In another embodiment said particles are fluorescently labeled beads, and the control sample is combined with the biofluid sample prior to contacting said biofluid sample with said affinity molecules. In another embodiment said control sample comprises positive control EV engineered to express the one or more of said target molecules exogenously, and optionally further comprises negative control EV that do not express said target molecules. In another embodiment said control sample comprises negative control EV obtained from non-neuronal cells that contain a first fluorescent marker, and positive control EV obtained from equivalent non-neuronal cells engineered to express the one or more of said target molecules, and containing a second, distinct fluorescent marker. In another embodiment the predetermined threshold corresponds to recovery of at least 44% of the predetermined amount of particles provided in the control sample.

In another embodiment the isolated EV are characterized by a ratio of neuron-specific marker levels to non-neuron-specific marker levels of at least 20-fold. In another embodiment said target molecules comprise NLGN3 and GAP43. In another embodiment said target molecules are NLGN3 and GAP43. In another embodiment said target molecules further comprise L1CAM and/or Rab3a. In another embodiment said target molecules are NLGN3, GAP43 and L1CAM. In another embodiment the substance is a plurality of magnetic beads. In another embodiment the affinity molecules are antibodies or comprise an antigen-binding portion thereof. In another embodiment the biofluid sample is selected from the group consisting of blood, plasma and serum. In another embodiment said subject is human.

In another aspect the method is used for diagnosing MCI in a subject in need thereof, comprising:
a. obtaining an EV-containing biofluid sample from the subject,
b. providing an isolation system comprising substance-bound affinity molecules capable of binding to target molecules on the surface of the EV, wherein at least one of the target molecules is a synaptic protein selected from the group consisting of NLGN3 and GAP43,
c. contacting the biofluid sample with said system, under conditions enabling specific binding of said affinity molecules to their corresponding target molecules,
d. isolating the EV bound to said affinity molecules,
e. determining the levels of Tau, p-Tau, Aβ42, and Neuroligin in the isolated EV, and
f. comparing the levels of Tau, p-Tau, Aβ42, and Neuroligin as determined in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with MCI.

In another embodiment the method further comprises:
i. providing a control sample containing a predetermined amount of particles that display one or more of the target molecules,
ii. contacting the control sample with the system, under the conditions enabling specific binding to their corresponding target molecules,
iii. quantifying the amount of particles bound by said affinity molecules, and
iv. determining that the amount of bound particles is over a predetermined threshold.

In another embodiment said particles are fluorescently labeled beads, and the control sample is combined with the biofluid sample prior to contacting said biofluid sample with said system. In another embodiment said control EV comprises positive control EV engineered to express the one or more of said target molecules exogenously, and optionally further comprises negative control EV that do not express said target molecules. In another embodiment said control sample comprises negative control EV obtained from non-neuronal cells that contain a first fluorescent marker, and positive control EV obtained from equivalent non-neuronal cells engineered to express the one or more of said target molecules, and containing a second, distinct fluorescent marker. In another embodiment the predetermined threshold corresponds to recovery of at least 44% of the predetermined amounts of particles provided in the control sample.

In another embodiment the isolated EV are characterized by a ratio of neuron-specific marker levels to non-neuron-specific marker levels of at least 20-fold. In another embodiment said target molecules comprise NLGN3 and GAP43. In another embodiment said target molecules are NLGN3 and GAP43. In another embodiment said target molecules further comprise L1CAM and/or Rab3a. In another embodiment said target molecules are NLGN3, GAP43 and L1CAM. In another embodiment the substance is a plurality of magnetic beads. In another embodiment the affinity molecules are antibodies or comprise an antigen-binding portion thereof. In another embodiment the biofluid sample is selected from the group consisting of blood, plasma and serum. In another embodiment said subject is human.

In other embodiments, the methods may be used for screening purposes, e.g. to evaluate whether a subject is at high risk for developing AD. Thus, in another aspect, there is provided a method of determining the likelihood of a subject to be predisposed to developing AD, comprising:
a. obtaining an EV-containing biofluid sample from the subject,
b. providing an isolation system comprising substance-bound affinity molecules capable of binding to target molecules on the surface of the EV, wherein at least one of the target molecules is a synaptic protein selected from the group consisting of NLGN3 and GAP43,
c. contacting the biofluid sample with said system, under conditions enabling specific binding of said affinity molecules to their corresponding target molecules,
d. isolating the EV bound to said affinity molecules,
e. determining the levels of -Tau, p-Tau, Aβ42, and Neuroligin in the isolated EV, and
f. comparing the levels of Tau, p-Tau, Aβ42, and Neuroligin as determined in the isolated EV to their respective levels corresponding to a control biofluid sample, to thereby compare the diagnostic signature of the sample to the control diagnostic signature, wherein a significant difference in the diagnostic signature of the biofluid sample compared to the control diagnostic signature indicates that said subject is predisposed to developing AD.

In another aspect the method is used for characterizing a FTD pathology in a subject in need thereof, comprising:
a. obtaining an EV-containing biofluid sample from the subject,
b. providing an isolation system comprising substance-bound affinity molecules capable of binding to target molecules on the surface of the EV, wherein at least one of the target molecules is a synaptic protein selected from the group consisting of NLGN3 and GAP43,
c. contacting the biofluid sample with said system, under conditions enabling specific binding of said affinity molecules to their corresponding target molecules,
d. isolating the EV bound to said affinity molecules,
e. determining the levels of TDP43 and p-Tau in the isolated EV,
f. comparing the levels of TDP43 and p-Tau as determined in the isolated EV to their respective control levels, and
g. characterizing the FTD pathology in said subject based on the levels of TDP43 and p-Tau determined in the isolated EV.

In another embodiment, enhanced levels of TDP43 and reduced levels of p-Tau in the isolated EV compared to control levels corresponding to a pool of FTD patients indicates that said FTD pathology is associated with a TDP43 proteinopathy, and
reduced levels of TDP43 and enhanced levels of p-Tau in the isolated EV compared to control levels corresponding to a pool of FTD patients indicates that said FTD pathology is associated with a Tau proteinopathy.

In another embodiment the method further comprises:
i. providing a control sample containing a predetermined amount of particles that display one or more of the target molecules,
ii. contacting the control sample with the system, under the conditions enabling specific binding to their corresponding target molecules,
iii. quantifying the amount of particles bound by said affinity molecules, and
iv. determining that the amount of bound particles is over a predetermined threshold.

In another embodiment said particles are fluorescently labeled beads, and the control sample is combined with the biofluid sample prior to contacting said biofluid sample with said system. In another embodiment said control sample comprises positive control EV engineered to express the one or more of said target molecules exogenously, and optionally further comprises negative control EV that do not express said target molecules. In another embodiment said control EV comprise negative control EV obtained from non-neuronal cells that contain a first fluorescent marker, and positive control EV obtained from equivalent non-neuronal cells engineered to express the one or more of said target molecules, and containing a second, distinct fluorescent marker. In another embodiment the predetermined threshold corresponds to recovery of at least 44% of the predetermined amounts of particles provided in the control sample.

In another embodiment the isolated EV are characterized by a ratio of neuron-specific marker levels to non-neuron-specific marker levels of at least 20-fold. In another embodiment said target molecules comprise NLGN3 and GAP43. In another embodiment said target molecules are NLGN3 and GAP43. In another embodiment said target molecules further comprise L1CAM and/or Rab3a. In another embodiment said target molecules are NLGN3, GAP43 and L1CAM. In another embodiment the substance is a plurality of magnetic beads. In another embodiment the affinity molecules are antibodies or comprise an antigen-binding portion thereof. In another embodiment the biofluid sample is selected from the group consisting of blood, plasma and serum.

In another embodiment said subject is human.

In another aspect, the method is used for diagnosing ALS in a subject in need thereof, and comprises:
a. obtaining an EV-containing biofluid sample from the subject,
b. providing an isolation system comprising substance-bound affinity molecules capable of binding to target molecules on the surface of the EV, wherein at least one of the target molecules is a synaptic protein selected from the group consisting of NLGN3 and GAP43,
c. contacting the biofluid sample with said system, under conditions enabling specific binding of said affinity molecules to their corresponding target molecules,
d. isolating the EV bound to said affinity molecules,
e. determining the levels of LC3, TDP43, and NRF2 in the isolated EV, and
f. comparing the levels of LC3, TDP43, and NRF2 as determined in the isolated EV to their respective levels corresponding to a control biofluid sample, to thereby compare the diagnostic signature of the sample to the control diagnostic signature,
   wherein a significant difference in the diagnostic signature of the biofluid sample compared to the control diagnostic signature indicates that said subject is afflicted with ALS.

In another aspect there is provided a method of diagnosing AD or predisposition thereto in a subject in need thereof, comprising:
a. obtaining an EV-containing biofluid sample from the subject,
b. providing an isolation system comprising substance-bound affinity molecules capable of binding to target molecules on the surface of the EV, wherein at least one of the target molecules is a synaptic protein comprising NLGN3 and GAP43,
c. contacting the biofluid sample with said system, under conditions enabling specific binding of said affinity molecules to their corresponding target molecules,
d. isolating the EV bound to said affinity molecules,
e. determining the levels of at least four biomarkers comprising at least one synaptic protein, at least one Tau gene product and at least one amyloid gene product in the isolated EV, and
f. comparing the levels of the biomarkers as determined in the isolated EV to their respective levels corresponding to a control biofluid sample, to thereby compare the diagnostic signature of the sample to the control diagnostic signature,
wherein a significant difference in the diagnostic signature of the biofluid sample compared to the control diagnostic signature indicates that said subject is afflicted with, or predisposed to developing, AD.

In another embodiment, the at least one synaptic protein is PSD95 or NLGN1, the at least one Tau gene product is Tau or p181-Tau, and the at least one amyloid marker is Aβ42. In another embodiment, said biomarkers are Aβ42, p-Tau, PSD95 and proBDNF, and a significant difference in the diagnostic signature of the biofluid sample compared to the control diagnostic signature indicates that said subject is afflicted with AD.

In another embodiment the method further comprises:
i. providing a control sample containing a predetermined amount of particles that display one or more of the target molecules,
ii. contacting the control sample with the system, under the conditions enabling specific binding to their corresponding target molecules,
iii. quantifying the amount of particles bound by said affinity molecules, and
iv. determining that the amount of bound particles is over a predetermined threshold.

In another embodiment said particles are fluorescently labeled beads, and the control sample is combined with the biofluid sample prior to contacting said biofluid sample with said system. In another embodiment said control sample comprises positive control EV engineered to express the one or more of said target molecules exogenously, and optionally further comprises negative control EV that do not express said target molecules. In another embodiment said control EV comprise negative control EV obtained from non-neuronal cells that contain a first fluorescent marker, and positive control EV obtained from equivalent non-neuronal cells engineered to express the one or more of said target molecules, and containing a second, distinct fluorescent marker. In another embodiment the predetermined threshold corresponds to recovery of at least 44% of the predetermined amounts of particles provided in the control sample.

In another embodiment the isolated EV are characterized by a ratio of neuron-specific marker levels to non-neuron-specific marker levels of at least 20-fold. In another embodiment said target molecules comprise NLGN3 and GAP43. In another embodiment said target molecules are NLGN3 and GAP43. In another embodiment said target molecules further comprise L1CAM and/or Rab3a. In another embodiment said target molecules are NLGN3, GAP43 and L1CAM. In another embodiment the substance is a plurality of magnetic beads. In another embodiment the affinity molecules are antibodies or comprise an antigen-binding portion thereof. In another embodiment the biofluid sample is selected from the group consisting of blood, plasma and serum. In another embodiment said subject is human. In another aspect there is provided a system for isolating neuron-derived EV, comprising means for identifying or capturing neuron-derived EV from a biofluid sample, comprising substance-bound affinity molecules capable of binding to target molecules on the surface of the EV, and further comprising (i) means for determining the accuracy of the system, and/or (ii) means for determining the levels of at least one biomarker in the captured EV,
wherein at least one of the target molecules is a synaptic protein (in particular a synaptic protein selected from the group consisting of NLGN3 and GAP43),
wherein the means for determining the accuracy of the system comprise particles that display the at least one synaptic protein and are labeled by a marker, and
wherein the at least one biomarker is selected from the group consisting of Tau, phosphorylated Tau (p-Tau), Aβ42, NLGN, TDP43, clusterin, SYP, BIM, NEFL, ENO2, NRGN, and GPR26 gene products, and combinations thereof, or selected from the group consisting of LC3, Cathepsin D, NRF2, Aβ42, p-Tau, PSD95, proBDNF, COX2, EIF2C2 and NF-κB and combinations thereof.

In another embodiment the target molecules comprise NLGN3 and GAP43. In another embodiment said target molecules are NLGN3 and GAP43. In another embodiment said target molecules are NLGN3, GAP43 and L1CAM. In another embodiment the means for determining the levels of at least one biomarker in the captured EV comprise antibodies directed to two, three, four, or five of said biomarkers. In another embodiment the means for determining the levels of at least one biomarker in the captured EV comprise antibodies directed to Tau, p-Tau, Aβ42, and Neuroligin-1. In another embodiment said means for determining the levels of at least one biomarker in the captured EV comprise antibodies directed to p-Tau and TDP43. In another embodiment said means for determining the levels of at least one biomarker in the captured EV comprise antibodies directed to Aβ42, p-Tau, PSD95 and proBDNF. In another embodiment said means for determining the levels of at least one biomarker in the captured EV comprise antibodies directed to LC3, TDP43, and NRF2.

In another aspect a kit for identifying or capturing neuron-derived EV is provided, comprising NLGN3-specific affinity molecules and GAP43-specific affinity molecules, capable of binding to NLGN3 and GAP43, respectively, on the surface of EV, and means for identifying or capturing the EV bound to the affinity molecules.

In another aspect the invention provides a kit for analyzing a biofluid sample of a subject suspected of having MCI, or of being predisposed to developing AD, comprising means for determining the levels of biomarkers in neuron-derived EV of the sample, wherein the biomarkers are Tau, p-Tau, Aβ42, and Neuroligin-1.

In another aspect, there is provided a kit for analyzing a biofluid sample of a subject suspected of having a neurological disorder, comprising means for determining the levels of biomarkers in neuron-derived EV of the sample, wherein:
the subject is suspected of having MCI, or of being predisposed to developing AD, and the biomarkers are Tau, p-Tau, Aβ42, and Neuroligin-1;
the subject is suspected of having AD, and the biomarkers are Aβ42, p-Tau, PSD95 and proBDNF; or the subject is suspected of having ALS, and the biomarkers are LC3, TDP43, and NRF2.

Other objects, features and advantages of the present invention will become clear from the following description and drawings.

### BRIEF DESCRIPTION OF THE DRAWINGS

FIG. 1A-1B. depicts NDE isolation using antibodies to various membrane proteins using a magnetic bead system. Total Tau (FIG. 1A) and p181-Tau (FIG. 1B) measured using a Luminex kit were used as neural protein markers.

FIG. 2. depicts NDE isolation using antibodies to various membrane proteins using a magnetic bead system, and quantification of neural mRNA markers by qPCR. Darker shades (lower Ct values) represent higher mRNA levels.

FIG. 3. shows specific recovery of exosomes isolated from human induced pluripotent stem cells (IPS)-derived neurons spiked into plasma samples.

FIG. 4A-4B. demonstrates the utilization of an engineered EV system for spike-in validation control. FIG. 4A—schematic illustration of the system. FIG. 4B, left panel—IgG control;

FIG. 4B, right panel—NDE isolation using an anti-NLGN3 antibody.

FIG. 5A-5F. demonstrates the utilization of a bead-based system for internal spike-in control. FIG. 5A—a schematic representation of the labeled beads; FIG. 5B—recovery rate (%) of labeled beads; FIG. 5C—Tau protein levels (pg/ml) in endogenous NDE isolated from the sample spiked-in with labeled beads; FIG. 5D—NRGN mRNA levels (Ct) in endogenous NDE isolated from the sample spiked-in with labeled beads; FIG. 5E—recovery curve for quantum dots (“QD input”-added QD; “QD recovery”—recovered QD; MFI—mean fluorescent intensity); FIG. 5F—Tau protein levels (pg/ml) in endogenous NDE isolated from the sample spiked-in with quantum dots (QD) and a sample without spike-in (no QD).

FIG. 6. demonstrates the use of a synergistic combinations of antibodies to multiple isolation targets providing highly selective NDE isolation. Results are presented as the fold change in the levels of the mRNA of neuronal marker NRGN (signal) relative to the mRNA level of the platelet marker PF4 (noise).

FIG. 7A-7B. show the isolation specificity using Western blot (WB) and fluorescence-activated cell sorting (FACS) analyses. FIG. 7A—WB on lysates of EV isolated by marker combination from two donors (NDE1 and NDE2), total plasma (P1) and HEK293 cell lysate (cell), measuring the levels of various neuronal and non-neuronal markers. The levels of the neuronal proteins L1CAM and GRIA2, general exosome markers ALIX, FLOT1, CD63, CD9 and CD81, and control proteins albumin and calnexin, were determined. FIG. 7B—exosomal (CD9) and neuronal (L1CAM) markers examined by FACS on intact EV isolated by marker combination (NDEs) or using a non-specific antibody (IgG control).

FIG. 8A-C. demonstrate the development of a highly accurate diagnostic classifier for Amyotrophic lateral sclerosis (ALS). FIG. 8A—Levels of LC3, Cathepsin D and TDP43 in ALS patients and control (n=35). FIG. 8B—Levels of COX-2, EIF2C2 and NRF2 (NFE2L2) in ALS patients and control (n=15). FIG. 8C—Receiver Operator Characteristic (ROC) curve based on combination of LC3, TDP43 and NFE2L2 levels for ALS patients vs control.

FIG. 9A-9C. illustrate the correlations between the levels of neural proteins in EV isolated using the antibody combination (NEVs) and brain tissue (cortex), in several animal models of Alzheimer's disease (AD). FIG. 9A—total Tau levels (tTau); FIG. 9B—phosphorylated Tau levels (p181-Tau); FIG. 9C—amyloid beta 42 levels (Aβ42). Circles represent wild-type mice (WT), squares represent 2×Tg-AD mice, crosses represent 3×Tg-AD mice, and stars represent 5×FAD mice.

FIG. 10A-B. demonstrate the development of a highly accurate diagnostic classifier for Mild Cognitive Impairment (MCI). FIG. 10A—levels of total Tau (tTau), phosphorylated Tau (pTau-181), amyloid beta 42 (Aβ42), amyloid beta 40 (Aβ401) and NLGN1 (NLGN) in individual patients (right) or healthy control subjects (left). FIG. 10B—diagnostic score combining the relative levels of Tau, p-Tau, NLGN and Aβ42, capable of identifying MCI patients.

FIG. 11A-H. show the levels of proteins in plasma neuron derived exosomes from early AD and healthy control. The levels were measured for Aβ42 (FIG. 11A), Tau (FIG. 11B), P-181-Tau (FIG. 11C), PSD95 (FIG. 11D), proBDNF (FIG. 11E), and NfkB (FIG. 11F). FIG. 11G—diagnostic score combining the relative levels of Aβ42, p-181-Tau, PSD95 and proBDNF in AD vs control. FIG. 11H—represents the identified score Receiver Operator Characteristic (ROC) curve.

FIG. 12. shows differential diagnosis of frontotemporal dementia (FTD) pathophysiology using multiple NDE markers.

### DETAILED DESCRIPTION OF THE INVENTION

The present invention relates to minimally invasive, biomarker-based diagnostics for neurodegenerative diseases, and to compositions and methods for isolating and analyzing specific populations of extracellular vesicles (EV). In particular, embodiments of the invention relate to methods and systems for isolating, identifying or capturing neuron-derived EV, for analyzing biological samples, and for diagnosing, assessing and predicting the development of neurological and neurodegenerative conditions.

The invention is based, in part, on the surprising identification of molecular targets including in particular synaptic proteins that are exceptionally useful for isolating neural-derived extracellular vesicles (NDE) from biological samples such as blood plasma and serum. It is herein disclosed for the first time, that neuroligin-3 (NLGN3) and growth-associated protein 43 (GAP43) are significantly more effective than other neuronal proteins, including the known target, L1 Cell Adhesion Molecule (L1CAM), as surface targets for capturing NDE in a selective manner. Surprisingly, it was discovered that a combination of substance-bound antibodies to NLGN3 and GAP43, either alone or further combined with a substance-bound anti-L1CAM antibody, acted in a synergistic manner to enhance the specific isolation capacity, and to improve the ratio of specific to non-specific markers as measured in the isolated EV. This unique combination provided a highly significant and proportional enrichment of brain proteins isolated from the captured EV, reflecting their levels in the tissue of origin. Interestingly, targeting NLGN3 and GAP43 in combination was even more selective than targeting NLGN3, GAP43 and L1CAM in combination, and further enhanced the ratio of specific (neuronal mRNA) to non-specific (platelet mRNA) marker levels measured in the isolated EV lysate.

The invention is further based, in part, on the development of improved analytical assays, providing reliable and consistent measurements amenable to clinical diagnostics. Remarkably, the use of validation spike-in controls based on engineered or manipulated EV, enabled evaluation of the specific EV recovery rate under a wide range of sample dilutions, and further established the applicability of the identified molecular targets for clinical use. In addition, a bead (microparticle)-based system was developed and determined particularly useful as an internal spike-in control, allowing direct evaluation of the recovery rate of each test sample, without significantly affecting the yield of the assay.

The invention is also based, in part, on the unexpected discovery of highly accurate diagnostic assays for neurological diseases, enabling early detection and characterization of the disease or pathology by analyzing peripheral blood samples. Using the improved NDE isolation methods and systems developed by the inventor, a number of diagnostic markers and signatures were identified, and found unexpectedly effective in evaluating the susceptibility, pathology and prognosis of human subjects with different neurodegenerative conditions. In particular, a highly accurate diagnostic classifier for Mild Cognitive Impairment (MCI, including MCI associated with early AD) based on a combination of four protein markers, and an assay for differential diagnoses of frontotemporal dementia pathophysiology based on a combination of two protein biomarkers, were unexpectedly developed. In addition, a remarkably accurate diagnostic classifier for Amyotrophic lateral sclerosis (ALS) based on a combination of only three biomarkers, and a diagnostic classifier for AD, combining the levels of four protein markers, were also developed using the systems and methods of the invention.

Thus, without wishing to be bound by a specific theory or mechanism of action, the isolation and evaluation of NDE that cross the blood brain barrier (BBB) into the blood circulation, using the improved methods and assays as disclosed herein, enables probing the central nervous system (CNS) neurons with high fidelity utilizing a simple blood test, without the need for performing a brain tissue biopsy. Further, the technical improvements as disclosed herein are advantageously compatible with large-scale applications, including robust and accurate assays and systems amenable for commercial, clinical-grade and high-throughput screening applications.

According to aspects and embodiments of the invention, provided are assays and methods in which neural derived exosomes or EV (NDE) are isolated, captured or selected from biological samples. According to embodiments of the invention, an EV-containing biological sample (including blood plasma or various other biofluid samples as disclosed herein) is contacted with one or more affinity molecules capable of specifically binding to synaptic protein targets on the surface of the NDE, and of providing enrichment for, or purification of, said NDE from the sample. In embodiments of the invention, the sample is contacted with the affinity molecules under conditions enabling specific binding of said affinity molecules to their corresponding targets (also referred to herein as target molecules or isolation markers). The NDE bound to the affinity molecules may then be obtained or selected from the sample, e.g. isolated and used for further analysis as disclosed herein. For example, various diagnostic markers may be detected on the surface of and/or within the isolated NDE, and their levels may be measured and compared to their respective control levels.

A. Isolation System and Related Assays

In some aspects and embodiments, the invention relates to an isolation system for EV, in particular for NDE. The isolation system in accordance with embodiments of the invention comprises substance-bound affinity molecules capable of binding to target molecules on the surface of the EV. Advantageously, as disclosed herein, the target molecules comprise at least one synaptic protein. In other embodiments, substance-bound affinity molecules capable of binding to target molecules on the surface of the EV may be used in assays and methods for identifying or capturing EV, in particular NDE.

Synaptic Proteins and Isolation Targets

As used herein, the term “synaptic proteins” refers to membrane proteins that are known to be significantly enriched in the synapse space, compared to non-synapse membrane areas. Synaptic proteins may be involved in regulation of neurotransmitter release and reception, holding the synaptic structure and/or in the early development of neurons.

In some embodiments synaptic proteins are disclosed herein to be superior targets for isolation or capture of NDE. In other embodiments, some synaptic proteins may also be used as diagnostic markers, for example in combination with other biomarkers to form diagnostic signatures, as will be described in further detail below.

In various embodiments, synaptic proteins useful as NDE isolation markers, either alone or in combination, include for example Neuroligin (NLGN) family members and/or counterpart ligands, thereof. As disclosed herein for the first time, NLGN3 is a particularly useful target for capturing NDE in a selective manner from non-neuronal biological fluids such as blood-derived samples. In additional embodiments, the use of other NLGN family proteins, including, but not limited to, NLGN1, or NLGN counterparts (ligands) including, but not limited to, neurexin (NRXN), is contemplated. In further embodiments, additional synaptic protein targets, including, but not limited to, GAP43, Synaptotagmin-1 (SYT1), and GRIA1 (Glutamate receptor 1, GluR1), may be used. Thus, according to additional embodiments, the invention employs the use of multiple targets (e.g. a plurality of synaptic protein targets as disclosed herein, or additional target molecules such as Rab3a). According to exemplary embodiments, substance-bound affinity molecules capable of binding to two, three or four target molecules on the surface of the EV may be used, wherein each possibility represents a separate embodiment of the invention.

Accordingly, in another exemplary embodiment, the invention relates to the use of at least one NLGN target in combination with additional synaptic or non-synaptic targets, e.g. NLGN3 combined with growth-associated protein 43 (GAP43) and optionally further combined with L1CAM, as target molecules for NDE isolation.

Thus, in another embodiment of the methods of the invention, the sample is contacted with (a combination of) affinity molecules capable of binding to the synaptic protein targets NLGN3 and GAP43 and to the additional target L1CAM, on the surface of the EV, under conditions enabling specific binding of the affinity molecules to their corresponding targets. In another particular embodiment of the methods of the invention, said affinity molecules are capable of binding to target molecules consisting of NLGN3 and GAP43 (e.g. antibodies directed to NLGN3 and GAP43, as explained and demonstrated below).

The details of certain preferable synaptic protein targets in accordance with embodiments of the invention are provided in Table 1 below. In some embodiments, one or more of these biomarkers may be used to capture or isolate NDE of human subjects according to the assays and methods as disclosed herein.

TABLE 1
Exemplary synaptic proteins

| Gene | Gene product | Description |
|---|---|---|
| GAP43 | GAP-43 | Growth-associated protein 43 (axonal membrane protein |
| | | that is also located in synapses). |
| NLGN3 | Neuroligin 3 | Structural synaptic protein |
| NLGN1 | Neuroligin 1 | Structural synaptic protein |
| NRGN | Neurogranin | Postsynaptic adaptor protein |
| GRIN2B | NMDAR2B | Glutamate Ionotropic Receptor NMDA Type Subunit 2B |
| GRIN3A | GluN3a | Glutamate Ionotropic Receptor NMDA Type Subunit 3A |
| GRIA1 | GluR1 | Glutamate Ionotropic Receptor AMPA Type Subunit 1 |
| GRIA2 | GluR2 | Glutamate Ionotropic Receptor AMPA Type Subunit 2 |
| SYP | Synaptophysin | synaptic vesicles protein (SYP, formerly designated SYP1) |
| SYT1 | Synaptotagmin 1 | synaptic vesicles protein |
| GRM1 | MGLUR1 | Glutamate Metabotropic Receptor 1 |
| GRM4 | MGLUR4 | Glutamate Metabotropic Receptor 4 |

Substance-Bound Affinity Molecules

In various embodiments, the affinity molecules include binding agents used to recognize the target of interest specifically, including, but not limited to, antibodies, receptors, ligands, small molecules, and aptamers. In some embodiments, the affinity molecule is an antibody or comprises an antigen-binding portion thereof.

An antibody directed (or specific) to an antigen, as used herein is an antibody which is capable of specifically binding the antigen. The term “specifically bind” or “specifically recognize” as used herein means that the binding of an antibody to an antigen is not competitively inhibited by the presence of non-related molecules.

Intact antibodies include, for example, polyclonal antibodies and monoclonal antibodies (mAbs). Exemplary functional antibody fragments comprising whole or essentially whole variable regions of both light and heavy chains (forming an antigen-binding portion) include, for example: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating whole antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating whole antibody with the enzyme pepsin, followed by reduction (two Fab′ fragments are obtained per antibody molecule); and (v) F(ab′)2, a fragment of an antibody molecule containing a monovalent antigen-binding portion of an antibody molecule, obtained by treating whole antibody with the enzyme pepsin (i.e., a dimer of Fab′ fragments held together by two disulfide bonds). Further included within the scope of the invention are chimeric antibodies; recombinant and engineered antibodies, single-chained antibodies (e.g. single-chain Fv) and fragments thereof (comprises the antigen-binding portion).

The term “antigen” as used herein is a molecule or a portion of a molecule capable of being bound by an antibody. The antigen is typically capable of inducing an animal to produce antibody capable of binding to an epitope of that antigen. An antigen may have one or more epitopes. The specific reaction referred to above is meant to indicate that the antigen will react, in a highly selective manner, with its corresponding antibody and not with the multitude of other antibodies which may be evoked by other antigens.

Methods of generating monoclonal and polyclonal antibodies are well known in the art. Antibodies may be generated via any one of several known methods, which may employ induction of in vivo production of antibody molecules, screening of immunoglobulin libraries, or generation of monoclonal antibody molecules by continuous cell lines in culture. These include, but are not limited to, the hybridoma technique, the human B-cell hybridoma technique, and the Epstein-Barr virus (EBV)-hybridoma technique. Besides the conventional method of raising antibodies in vivo, antibodies can be generated in vitro using phage display technology, by methods well known in the art (e.g. Current Protocols in Immunology, Colligan et al (Eds.), John Wiley & Sons, Inc. (1992-2000), Chapter 17, Section 17.1).

Non-limitative examples of antibodies directed to NLGN3 include e.g. LSBio LC-C819103, SinoBiological 11160-R242, RayBiotech 101-11116, Novus MBP2-89824, Abnova H00054413-MO2, Rockland 200-306-W83, Biorbyt orb416408, Boster A04627-1, and G-Biosciences ITA9433. Non-limitative examples of antibodies directed to GAP43 include e.g. Biorbyt orb621812, LsBio LS-B2157, HuABIo ET1610-94, Abnova MAB20070, H00002596-M01, ThermoFisher MA5-32256, ThermoFisher 33-5000, ABCAM ab75810, MyBioSource MBS179756, MyBioSource MBS243028, and OriGene RA25086-100.

In another embodiment, the affinity molecule is an aptamer specific to a target molecule as disclosed herein, e.g. a nucleic acid aptamer or a peptide aptamer. The term “aptamer” refers to a special kind of single-stranded nucleic acid, double-stranded nucleic acid, or peptide that has a stable tertiary structure and can bind to a target molecule with high affinity and specificity. In particular embodiments, the aptamer may be DNA, RNA, or a combination thereof, but is not limited thereto. In addition, the aptamer may be unmodified, that is, a natural aptamer or a modified aptamer. Specifically, the modified aptamer may include at least one chemical modification, and the at least one chemical modification is chemical at one or more positions independently selected from ribose position, deoxyribose position, phosphate position, and base position. In addition, the chemical modification may be 2′-position sugar modification, 2′-fluoro (2′-F), 2′-O-methyl, purine modification at 8-position, cytosine exo Modification in exocyclic amines, substitution of 5-bromouracil, substitution of 5-bromodeoxyuridine, substitution of 5′-bromodeoxycytidine (5-bromodeoxycitidine) substitution, backbone modification, methylation, 3′ cap (3′ cap) and/or 5′-cap (5′ cap). In addition, the hydrophobic functional group may be at least one selected from the group consisting of a benzyl group, a naphthyl group, a pyrrolebenzyl group, and tryptophan. Peptide aptamers are artificial proteins selected or engineered to bind specific target molecules. These proteins consist of one or more peptide loops of variable sequence displayed by a protein scaffold. Aptamers can be prepared in vitro through e.g. SELEX (Systematic evolution of ligands by exponential enrichment), or using libraries of peptide aptamers as known in the art (e.g. the yeast two-hybrid system).

In another embodiment, the affinity molecule is a ligand that interacts with the synaptic protein. As used herein, a ligand is defined as a protein or a peptide that specifically interacts with the target protein (e.g. receptor). In the context of affinity molecules directed to synaptic proteins as target molecules (e.g. NLGN ligands), ligands are defined as the membrane proteins located on the opposing part of the synapse (e.g. presynaptic in the case of a postsynaptic target, and vice a versa) and interact with said target as part of the synaptic structure. For example, NLGN3 interacts with NRXN1, NRXN2 and NRXN3 as well as with PIC3R1 and PIC3R2. These proteins, or a fragment thereof which mediates the interaction with NLGN3 can be synthesized and immobilized on beads and use as an affinity molecule targeting NLGN3. The synthesis of such a protein or peptide can be done by few methods that are known in the art. For example, by expression in E. coli, other bacteria, HEK293 cells or other cell type. Short peptides can also be synthesized using solution phase synthesis (SPS), solid phase peptide synthesis (SPPS), or other common method of synthesis. As another example, GAP43 interacts with CALM1, CALM2, NCAM. Calmodulin, PRKCB and ELAVL4. These can be used as full proteins, or as partial peptides. The ligand proteins can be used in their natural form or following improvement using methods that are known in the art. For example, improvement of the interaction can be obtained by artificial evolution, random or direct maturation, substitution to non-natural amino acids and other methods known to change and select improve binders.

In another embodiment, said affinity molecule is bound to a substance, to form a substance-bound affinity molecule. For example, the affinity molecules (e.g. antibodies) may be bound to the outer surface of various substances, typically solid or semisolid substances enabling the capture or isolation of the bound EV. Examples of suitable substances include, but are not limited to, beads (including in particular magnetic beads or particles), nanoparticles, columns, plates, microfluidic structures, liposomes, or combinations thereof. As used herein, the term “substance-bound” denotes that the affinity molecule is attached to the substance by covalent conjugation or other forms of chemical links that are stable under the conditions used in the methods of the invention, such that isolation, capture or identification of EV is facilitated. In some embodiments, the affinity molecule may be attached to the substance by biotin-streptavidin interaction or the like. For example, the affinity molecule may be an antibody bound to a biotin molecule, or a biotin-containing linker (including cleavable linkers), and the substance may be bound to or coated with biotin counterparts such as streptavidin or avidin molecules. In a particular embodiment, said substance is streptavidin-conjugated magnetic microparticles. In other embodiments, the affinity molecule is covalently bound to the substance (either directly or via a linker, using suitable chemical groups including, but not limited to, primary amines, sulfhydryls, carboxylic acids, and aldehydes), or attached to the substance by protein-protein interaction. Each possibility represents a separate embodiment of the invention.

As used herein, the term “magnetic beads” refers to a nano- or micro-scale particle that is attracted or repelled by a magnetic field gradient or has a non-zero magnetic susceptibility. The magnetic bead can be paramagnetic or super-paramagnetic. In some embodiments, magnetic beads are super-paramagnetic. Magnetic beads are also referred to as magnetic particles herein.

The magnetic beads can range in size from 1 nm to 1 mm. For example, magnetic beads are about 250 nm to about 250 μm in size. In some embodiments, magnetic bead is 0.1 μm to 100 μm in size, 0.1 μm to 50 μm in 0.1 μm to 10 μm in size, 1 μm to 10 μm in size, 2 μm to 7 μm in size, or 1 μm to 3 μm in size. In some embodiments, the magnetic bead is a magnetic nano-particle or magnetic micro-particle. Magnetic nanoparticles are a class of nanoparticle which can be manipulated using magnetic field or magnetic field gradient. Such particles commonly consist of magnetic elements such as iron, nickel and cobalt and their chemical compounds. Magnetic nano-particles are well-known and methods for their preparation have been described in the art. See, e.g., U.S. Pat. Nos. 6,878,445; 5,543,158; and 5,578,325.

Magnetic beads are available commercially, with or without functional groups capable of binding to affinity molecules, for example from Dynal Inc. (Lake Success, N.Y.); PerSeptive Diagnostics, Inc. (Cambridge, Mass.); Invitrogen Corp. (Carlsbad, Calif.); Cortex Biochem Inc. (San Leandro, Calif.); and Bangs Laboratories (Fishers, Ind.).

In a particular embodiment, the isolation system comprises antibodies directed to isolation targets as disclosed herein, which are chemically linked to superparamagnetic microparticles. For example, without limitation, the substance used as a carrier for the affinity molecules during the isolation process may contain superparamagnetic microparticles (beads) of 2-10 μm comprised of iron (e.g. iron oxide) and optionally one or more polymers. The surface of these particles may be coated with functional groups or elements (including, but not limited to, streptavidin or biotin) enabling conjugation of the antibodies or other affinity molecules. The system may further contain a magnet which is conveniently adapted for use with a tube rack.

In other embodiments, the substance may comprise a column, in particular an affinity column. The column can contain different affinity matrices, including but not limited to agarose, cellulose, dextran, polyacrylamide, latex and controlled pore glass. In other embodiments, the column can contain a gel support, including but not limited to sugar, agarose and acrylamide-based polymer resins in various densities (2-10%). The affinity column can be of various sizes, and work with gravity or pumps of different kinds. The affinity molecules can be immobilized to the column using covalent chemical interaction (e.g. via primary amine, sulfhydryl, carboxylic acid, or aldehyde residues) or non-covalent interaction.

In additional embodiments, the substance can be a plate or a tube. The plate can be of different sizes including but not limited to 384, 96, 24, 12 and 6-well plates. The affinity molecules can be immobilized to the column using covalent chemical interaction (e.g. via primary amine, sulfhydryl, carboxylic acid, or aldehyde residues) or non-covalent interaction such as charge or hydrophobic interaction. The use of the plate or tube can be done alone or together with other instrument that shake or rotate the plate as well as circulating the liquid.

In yet other embodiments, the substance can be a microfluidic device. The device can be a standalone, meaning the affinity molecule being directly attached to the microfluidic device, or it can work in combination with beads or other substances as described herein. The microfluidic substance can be fabricated from silicon, glass, polymers (e.g. polyvinylchloride), composites, or paper. The device structure can be design according to basic criteria of microfluidic flow known to a person in the art. Examples for microfluidic technology that have been used for exosome isolation and may be combined with embodiments of the invention are reviewed by Lliescu et al. (Micromachines (Basel). 2019 June; 10(6): 392).

Binding and Isolation Steps and Conditions

According to embodiments of the invention, the sample is contacted with the affinity molecules under conditions enabling specific binding of the affinity molecules to their corresponding targets. In some embodiments, the conditions allow the formation of specific antigen-antibody complexes. For example, contacting may be performed under physiological conditions or other conditions (e.g. specific ranges of temperature, pH, ionic strength etc.) known in the art as suitable for performing immunoassays, e.g. as described and exemplified hereinbelow. In another exemplary embodiment, incubation may be performed with rotation or shaking, in a range of temperatures of above 0° C. and below 60° C., e.g. 4-40° C. By means of non-limitative examples, the conditions may include incubation with slow rotation (10-100 rotations per minute (rpm)) or shaking (300-700 rpm), at a range of temperatures of between 4-50° C., for 1-24 hours. The incubation may advantageously be performed in the presence of protease inhibitors and optionally also phosphatase inhibitors.

In some embodiments, washing and/or blocking steps may be further performed in order to eliminate or minimize non-specific binding of unrelated molecules. According to exemplary embodiments, washing may be performed (e.g. after binding the affinity molecules to the substance, after blocking the affinity molecules with buffers as described herein and/or after incubating the sample with the substance-bound affinity molecules), with buffers having suitable ranges of pH (e.g. 6-8), salt concentrations (e.g. 0.1-1M) and detergent content (e.g. 0.1-1%), depending on the system to be used. In other exemplary embodiments, blocking the affinity molecules (prior to incubation with the sample) may be conveniently performed with buffers containing BSA (e.g. 1-10%), milk (e.g. 1-10%), ethanolamine (e.g. 10-100 mM), biotin (e.g. 1-50 uM) or combinations thereof, depending on the system to be used.

As used herein, “isolation” of EV refers to their physical separation from their environment (e.g. biofluid sample), to obtain a substantially pure or enriched EV population. Identification and capturing of EV include various methods which enable obtaining information on the nature and/or quantity of a specific EV population, without necessarily involving physical separation from the substance or their environment. Such methods may further involve a step of EV isolation, or may facilitate quantification or identification directly from the substance. Identification methods can utilize the same affinity molecules used for isolation. According to exemplary embodiments, a subsequent step of EV isolation, involving e.g. elution of said EV from the substance-bound affinity molecules under conditions involving e.g. elevated detergent content and/or reduced pH, is included. In other exemplary embodiments, such an isolation step is not included, and the EV remain intact and bound to the substance-bound affinity molecules. Identification of the EV may then be performed by evaluating the amount of bound EV, e.g. using a second affinity molecule directed to an EV marker (e.g. antibodies directed to one or more general EV markers such as CD9, CD63 or CD81) or a color agent that interacts directly with EV membranes.

EV Populations

In some embodiments, the invention relates to an isolated EV population, obtained by a method as disclosed herein. In another embodiment, the EV population comprises, or consists essentially of, NDE. As used herein, the term “extracellular vesicles” (EV) refers to double membrane vesicle (20 nm to 1 μm in size) of various biogenesis pathways, and includes both vesicles released from living cells and from dying cells. In some embodiments, EV populations isolated by the methods of the invention typically comprise exosomes. Thus, in the context of the methods of the invention and unless indicated otherwise, the terms “exosomes” and “EV” may be used interchangeably.

As used herein, the term “NDE” refers to an EV population derived from (secreted by) one or more neural cell populations, that may be isolated from various biofluids (e.g. blood, plasma and serum). In some embodiments, NDE as referred to in connection with diagnostic methods of the invention are further defined as being distinguished from EV that are secreted from cell cultures in vitro (e.g. from differentiated neural cells). In a particular embodiment, the NDE are isolated from blood or blood-derived samples.

B. Processing and Analysis

Samples

In various embodiments, the EV-containing sample, also referred to herein as a biofluid sample or a biological sample, is a bodily fluid of the subject or another liquid sample, including, in particular, blood-derived samples (e.g. plasma or serum). In some embodiments, other biofluid samples, including, but not limited to CSF samples and saliva samples, may be used. In a particular embodiment, the biofluid sample is other than a CSF sample. In various other embodiments, additional biological samples including, but not limited to, conditioned media of cultured neurons, may be used. In various embodiments, is obtained from a subject afflicted with, or suspected of having or being predisposed to, a disease or disorder as detailed hereinbelow.

In some embodiments, the NDE isolation or identification is performed directly from the biofluid sample (e.g. using non-manipulated or diluted plasma samples). In yet other embodiments, one or more additional steps of manipulation may be performed to enrich the sample with EV. For example, without limitation, enriching for total EV (non-tissue-specific EV) may be performed by centrifugation, filtration, charge separation, chemical precipitation and combinations thereof, either before or after the affinity selection. For example, as exemplified herein, a sample obtained from cultured neurons is of large volume (10-400 ml) and low EV abundance and thus may be processed by size exclusion chromatography, anion exchange chromatography and/or ultrafiltration prior to subjecting said sample to specific isolation or capturing steps in accordance with the invention.

Biomarkers and Diagnostic Markers

According to additional embodiments, the sample is further analyzed by detection and/or quantification of the EV payload, e.g. of proteins, RNA, lipids and/or metabolites of the isolated NDE (e.g. either expressed on the surface of intact NDE or present in the NDE lysate). In some embodiments, the methods of the invention include measuring the levels of one or more EV biomarkers, which may be associated with various diseases and conditions, thereby serving as diagnostic or prognostic markers. According to embodiments of the invention, the levels of one or more biomarkers in said EV are measured and compared to their respective levels corresponding to (EV of) a control biological sample. In some embodiments, the methods include measuring the levels of a plurality of biomarkers and determining the resulting diagnostic signature, which may be compared to a control signature (corresponding to EV of a control sample).

As used herein, the term “biomarker” refers to a distinctive biological or biologically derived indicator of a process, event, or condition. Biomarkers as used herein encompass, without limitation, gene products, including proteins, nucleic acids, and metabolites, together with their polymorphisms, mutations, variants, modifications, subunits, fragments, protein-ligand complexes, and degradation products, and other analytes (e.g. lipids and sugars) or sample-derived measures that are associated with a biological state. In one embodiment, the biomarker is indicative of the origin of the cell or tissue from which the EV are derived (e.g. neuron-specific markers and platelet-specific markers as exemplified herein).

The term “diagnostic marker” refers to a biomarker that is associated with a specific disease, disease state, pathology or diagnosable condition in a subject. In particular, diagnostic markers in accordance with embodiments of the invention include gene products in which their differential expression (or post-translationally altered levels) in individuals with a disease or condition compared to control individuals, facilitate diagnosis, prognosis and/or monitoring of the disease or condition.

In various embodiments, the biomarkers may include, but are not limited to, neural proteins such as Microtubule Associated Protein Tau (Tau, total protein and/or phosphorylated fraction), Amyloid-beta 42 (Aβ42), Neuroligin (NLGN, including in particular NLGN1), TAR DNA-binding protein 43 (TDP43), neurogranin (NRGN), SYP, Cathepsin D, LC3, SYT, BIM, NEFL, ENO2, GPR26 and combinations thereof, as discussed in further detail and exemplified below. In another embodiment the one or more biomarkers are selected from the group consisting of Tau, p-Tau, Aβ42, and NLGN, TDP43, CLU, SYP, BIM, NEFL, ENO2, NRGN, and GPR26 gene products, and combinations thereof. Each possibility represents a separate embodiment of the invention. In a particular embodiment, phosphorylated Tau includes Thr-181-phosphorylated Tau (p181-Tau).

In one embodiment, the biomarker is selected from the group consisting of: LC3, Cathepsin D, NRF2, Aβ42, p-Tau, PSD95, proBDNF, COX2, EIF2C2 and NF-κB. In other embodiments, the biomarkers may be selected from the group consisting of: total-Tau, p-Tau, Aβ42, PSD95, proBDNF and NFκB. In other embodiments, the biomarkers may include total-Tau, p-Tau, Aβ42, PSD95, proBDNF and NFκB. In another embodiment said biomarkers are Aβ42, p-Tau, PSD95 and proBDNF. In another embodiment the biomarker is selected from the group consisting of LC3, TDP43, NRF2 (NFE2L2), cathepsin D, COX2 and EIF2C2. In yet other embodiments, the biomarker may be selected from the group consisting of LC3, TDP43, and NRF2. In other embodiments, the biomarkers may include LC3, TDP43, and NRF2.

The details of certain preferable biomarkers in accordance with embodiments of the invention are provided in Table 2 below. In some embodiments, one or more of these biomarkers (gene products, at either the transcript or the protein level) may be detected or quantified in NDE of human subjects isolated or captured as disclosed herein, for use in diagnosis of various neurological conditions as disclosed herein. Additional non-limitative examples of biomarkers that may be assayed in accordance with embodiments of the invention are presented and exemplified in Table 5 (lipid biomarkers), as well as throughout the Examples section.

TABLE 2
Exemplary biomarkers

| | Gene | |
|---|---|---|
| | product/ | |
| Gene | biomarkers | Description |
| MAPT | Tau, total-Tau | Microtubule Associated Protein Tau, phosphorylated and non- |
| | | phosphorylated |
| | p-Tau | Phosphorylated Tau (p181-Tau, p231-Tau, p217-Tau, p396-Tau), |
| | | in particular p181-Tau |
| NLGN1 | Neuroligin 1 | Structural synaptic protein |
| NRGN | Neurogranin | postsynaptic adaptor protein |
| SYP | Synaptophysin | synaptic vesicles protein |
| HTR2A | 5-HT2A | 5-Hydroxytryptamine Receptor 2A |
| TARDBP | TDP-43 | TAR DNA Binding Protein |
| APP | Aβ40, Aβ42, | Amyloid Beta (A4) Precursor Protein and its processed amyloid |
| | CTFβ | beta (Aβ) peptides |
| MAP1LCB | LC3B | Microtubule Associated Protein 1 Light Chain 3 Beta |
| MAP1LCA | LC3 | Microtubule Associated Protein 1 Light Chain 3 Alpha |
| CTSD | Cathepsin D | Lysosomal protease |
| NFE2l2 | NRF2 | Nuclear Factor, Erythroid 2 Like 2 |
| BAD | BAD | BCL2 Associated Agonist of Cell Death (phosphorylated and non- |
| | | phosphorylated) |
| | p-BAD | Phosphorylated BAD |
| DLG4 | PSD95 | Discs Large MAGUK Scaffold Protein 4 (synaptic protein) |
| BDNF | proBDNF and | Brain-Derived Neurotrophic Factor |
| | mature BDNF | |
| MT-CO2 | COX2 | Mitochondrially Encoded Cytochrome C Oxidase II |
| AGO2 | EIF2C2 | Argonaute RISC Catalytic Component 2 |
| NFKB1, NFKB2 | NF-κB | Nuclear factor kappa-light-chain-enhancer of activated B cells |
| LAMP2 | LAMP-2 | Lysosomal Associated Membrane Protein 2 |
| CLU | Clusterin | Multi-functional protein, genetic risk for AD |
| BCL2L11 | BIM | Bcl-2 Interacting Mediator of Cell Death (apoptosis regulating |
| | | protein) |
| NEFL | NFL | Neurofilament Light Chain (neuron-specific marker; marker of |
| | | axon damage) |
| ENO2 | Enolase 2 | Metabolism protein (neuron-specific marker) |
| GPR26 | GPR26 | G Protein-Coupled Receptor 26 (neuron-specific marker; linked to |
| | | depression disorder) |

Neuroligin (NLGN), a type I membrane protein, is a cell adhesion protein on the postsynaptic membrane that mediates the formation and maintenance of synapses between neurons. Neuroligins act as ligands for β-Neurexins (NRXN), which are cell adhesion proteins located presynaptically. Neuroligin and β-neurexin bind specifically, resulting in the connection between two neurons and the production of a synapse. Neuroligins also affect the properties of neural networks by specifying synaptic functions, and they mediate signaling by recruiting and stabilizing key synaptic components. Neuroligins interact with other postsynaptic proteins to localize neurotransmitter receptors and channels in the postsynaptic density as the cell matures. When used as a diagnostic biomarker in the methods and assays of the invention, the term “Neuroligin” or “NLGN” denotes in particular a NLGN gene product comprising NLGN1, or immunologically cross-reactive with NLGN1.RNA biomarkers may be detected or quantified, for example, using various RNA analysis methods including, but not limited to, RT-PCR, qPCR, ddPCR, RNA-seq, chip array and the like, and protein biomarkers may be detected or quantified using various immunoassays or other protein analysis methods e.g. ELISA, protein array, mass spectrometry, flow cytometry and the like. Such assays are discussed in greater detail below. Non-limitative examples for detected and quantified biomarkers are provided in the Examples section below.

Biomarker Detection and Quantification

In some embodiments, the methods of the invention involve determining the levels of biomarkers in the isolated or bound EV. The levels determined may be used to evaluate if the biomarker is present in the sample (detection) and/or to evaluate its absolute or relative amount (quantification). Various methods for determining the levels of biomarkers, including, but not limited to, gene products at their mRNA and/or protein levels, are known in the art.

According to some embodiments, suitable methods to detect and/or quantify mRNA biomarkers may include such methods as, but not limited to: amplification reaction, comprising, e.g., polymerase chain reaction (PCR), reverse-transcriptase PCR (RT-PCR, e.g. quantitative or semi-quantitative), real time PCR, and the like; Gene expression microarrays, such as Affymetrix, Agilent, and Illumina microarray platforms; NanoString technology, Sequencing, such as, next Generation Sequencing (NGS) using specific adaptors and/or probes; and the like, or combinations thereof.

For example, real time quantitative PCR (RT-qPCR) allows amplification and simultaneous quantification of a target DNA molecule. To analyze gene expression levels using RT-qPCR, the total mRNA of a sample (e.g. an isolated NDE population as disclosed herein) may first be isolated and reverse transcribed into cDNA using reverse transcriptase. For example, mRNA levels can be determined using e.g. TaqMan Gene Expression Assays (Applied Biosystems) on an ABI PRISM 7900HT instrument according to the manufacturer's instructions. Transcript levels can be compared between samples in a semi-quantitative manner according to the well-established delta cycle threshold (ΔCt) method, or abundance can be calculated by comparison to a standard curve.

Digital PCR is a new approach to nucleic acid detection and quantification that offers an alternate method to conventional real-time quantitative PCR for absolute quantification and rare allele detection. Digital PCR works by partitioning a sample of DNA or cDNA into many individual, parallel PCR reactions; some of these reactions contain the target molecule (positive) while others do not (negative). A single molecule can be amplified a million-fold or more. During amplification, TaqMan chemistry with dye-labeled probes is used to detect sequence-specific targets. When no target sequence is present, no signal accumulates. Following PCR analysis, the fraction of negative reactions is used to generate an absolute count of the number of target molecules in the sample, without the need for standards or endogenous controls. The use of a nanofluidic chip provides a convenient and straightforward mechanism to run thousands of PCR reactions in parallel. Each well is loaded with a mixture of sample, master mix, and TaqMan Assay reagents, and individually analyzed to detect the presence (positive) or absence (negative) of an endpoint signal. To account for wells that may have received more than one molecule of the target sequence, a correction factor is applied using the Poisson model. Droplet Digital PCR (ddPCR) has been used for transcripts with very low abundance.

RNA-SEQ uses next-generation sequencing (NGS) for the detection and quantification of RNA in a biological sample at a given moment in time. An RNA library is prepared, transcribed, fragmented, sequenced, reassembled and the sequence or sequences of interest quantified. NanoString technology uses unique color-coded molecular barcodes that can hybridize directly to many different types of target nucleic acid molecules, and offers a cost-effective way to analyze the expression levels of up to 800 genes simultaneously, with sensitivity comparable to qPCR.

For microarray analysis of e.g. gene expression, total RNA is first obtained from the isolated EV population using, for example, Trizol or an RNeasy mini kit (Qiagen). The isolated total RNA is then reverse transcribed into double-stranded cDNA using reverse transcriptase and polyT primers and labelled using e.g. Cy3- or Cy5-dCTP. Appropriate Cy3- and Cy5-labelled samples are then pooled and hybridized to custom spotted oligonucleotide microarrays comprised of probes representing suitable genes and control features. Samples may be hybridized in duplicate, using a dye-swap strategy, against a common reference RNA. Following hybridization, arrays are washed and scanned on e.g. an Agilent G2565B scanner. Suitable alternatives to the steps described above are well known in the art and would be apparent to the skilled person. The raw microarray data obtained can then be analyzed using suitable methods to determine the relative expression of genes of interest, as applicable.

In certain embodiments, methods of the invention are performed by an immunoassay, using antibodies (or other affinity molecules) specific to one or more biomarkers of the invention (e.g. gene products or lipid biomarkers). In various embodiments, the immunoassay is selected from the group consisting of dipstick, ELISA (including various multiplexed ELISA technologies), an antibody array, an antibody chip, a lateral flow test, and multiplex bead immunoassay.

In accordance with the principles of the invention, any suitable immunoassay can be used. Such techniques are well known to the ordinarily skilled artisan and have been described in many standard immunology manuals and texts. In certain embodiments, determining the levels of gene products is performed using an antibody array-based method, including, but not limited to an antibody array or an antibody chip. In some embodiments, the array is incubated with an optionally diluted sample (e.g NDE or NDE lysate) of the subject so as to allow specific binding between the gene products contained in the sample and the immobilized antibodies, washing out unbound components from the array, incubating the washed array with detectable label-conjugated antibodies of the desired isotype, washing out unbound label from the array, and measuring levels of the label bound to each gene product.

In certain embodiments, the detection of the biomarkers (gene products) may be performed using other immunoassays such as an enzyme-linked immunosorbent assay (ELISA) testing kit. In such assays, for example, samples are typically incubated in the presence of an immobilized first specific binding agent (e.g. an antibody) capable of specifically binding the biomarker. Binding of the biomarker to said first specific binding agent may be measured using any one of a variety of known methods, such as using a labeled second specific binding agent capable of specifically binding the biomarker (at a different epitope) or the first specific binding agent. Exemplary specific binding agents include e.g. monoclonal antibodies, polyclonal antibodies, and antibody fragments such as recombinant antibody fragments, single-chain antibodies (scFv) and the like. In some embodiments, various conventional tags or labels may be used, such as a radioisotope, an enzyme, a chromophore or a fluorophore. A typical radioisotope is iodine⁻¹²⁵ or sulfur⁻³⁵. Typical enzymes for this purpose include horseradish peroxidase, horseradish galactosidase and alkaline phosphatase. Conveniently, other detection methods involving chemiluminescence, electrochemiluminescence (ECL) or fluorescence, may readily be used for evaluating the amount of bound detection antibody.

Lateral flow tests operate on the same principles as ELISA assays as described above. In essence, these tests run the sample along the surface of a pad with reactive molecules that show a visual positive or negative result. The pads are based on a series of capillary beds, such as pieces of porous paper, microstructured polymer, or sintered polymer. Each of these pads has the capacity to transport fluid spontaneously. The sample pad acts as a sponge and holds an excess of sample fluid. Once soaked, the fluid flows to the second conjugate pad in which freeze dried bio-active particles called conjugates are stored in a salt-sugar matrix. The conjugate pad contains all the reagents required for an optimized chemical reaction between the target molecule (e.g., a gene product as disclosed herein) and its chemical partner (e.g., antibody) that has been immobilized on the particle's surface. This marks target particles as they pass through the pad and continue across to the test and control lines. The test line shows a signal, often a color. The control line contains affinity ligands which show whether the sample has flowed through and the bio-molecules in the conjugate pad are active. After passing these reaction zones, the fluid enters the final porous material, the wick, that simply acts as a waste container.

Additional exemplary assays may be based on dipstick technology, as demonstrated, for example, in U.S. Pat. Nos. 4,632,901, 4,313,734 and 4,786,589 5,656,448 and EP 0125118. Alternately, other immunoassays may be used; such techniques are well known to the ordinarily skilled artisan and have been described in many standard immunology manuals and texts. In some embodiments, the methods of the invention are suitable for automated or semi-automated analysis, and may enable clinical, medium or high-throughput screening of multiple samples. For example, automated ELISA systems such as Biotest's Quickstep® ELISA Processor, Maxmat Automated microwell ELISA analyzer (Maxmat S. A., France), or DSX™ Four-Plate System (Dynex Technologies) may conveniently be used, and employed in various methods including, but not limited to multiplexed ELISA methods. Other suitable assays include for example flow cytometry assays (such as singleplex and multiplex bead-based Luminex® assays (Invitrogen), or other multiplex bead immunoassays available in the art.

In another embodiment, determining the levels of said gene products is performed by mass spectrometry or using a micro-spectrometer. For example, using heavy labeled synthetic internal standards for the proteolytic peptides of said gene products. The native peptides and the standards may be measured using a mass spectrometer and the signal from the internal standard is referenced to the native peptides, which represent the original protein in the sample. In a non-limitative example, suitable equipment such as the SCIO Near Infrared Mini-Spectrometer may be used.

C. Quality Control and Accuracy Determination

In some embodiments, the methods and assays of the invention employ the use of validation or spike-in controls, enabling system calibration and quality control, and providing improved accuracy and quantitative measurements. In various embodiments, the methods and systems of the invention include the advantageous use of engineered, labeled and/or experimentally-induced exogenous EV controls, which may be produced and selected for use according to the system parameters to be tested and markers to be used. In other embodiments, the invention employs the use of target-specific labeled particles, that may be supplemented to the samples and serve as internal controls. Thus, the invention in advantageous embodiments thereof provides a significant improvement in quality assurance and control, and in the ability to limit assay variability due to experimental parameters and sample-processing issues.

Thus, according to aspects and embodiments of the invention, provided is a control sample containing a predetermined amount of particles that display one or more of the target molecules that are displayed on the surface of the EV that are intended to be isolated, captured or detected. In various embodiments, the particles may be vesicles (e.g. EV) or non-vesicle particles (e.g. synthetic micro- or nano-beads). In a particle displaying a target, the target molecules are presented on its outer surface, in a manner enabling their specific binding by corresponding affinity molecules (e.g. antibodies).

Control EV

In some embodiments, the controls comprise positive control EV (e.g. transfected with or otherwise induced to express a synaptic protein target selected as an isolation marker), used at a predetermined amount, enabling evaluation of the ability of the system to isolate EV expressing the target. Additionally or alternatively, the controls may comprise negative control EV, enabling evaluation of the degree of contamination by non-related vesicles or particles. For example, a control sample may be supplemented (spiked-in) with positive control EV and negative control EV at predetermined amounts (and ratio), and the specific recovery rate of the positive control EV to negative control EV may be determined.

In some embodiments, the positive control EV are obtained from cells engineered to express exogenously a target molecule, such as a synaptic protein target or a combination of target molecules as disclosed herein, and the negative control EV are obtained from equivalent cells that do not naturally express the target (or that express it at a lower basal level). Thus, the control EV may be contacted with the affinity molecules under the same conditions enabling specific binding of said affinity molecules to their corresponding targets, and isolated accordingly. Conveniently, the positive control EV and the negative control EV are labeled with e.g. fluorescent molecules or other suitable labels or dyes (also referred to herein as markers) that facilitate EV identification or isolation. For example, without limitation, the negative control EV may be obtained from non-neuronal cells (that may contain a first label, e.g. a membrane-staining fluorescent dye such as PKH26), and the positive control EV may be obtained from the same (or equivalent) non-neuronal cells engineered to overexpress NLGN3, or another target or target combination of the invention, and advantageously contain a second label (e.g. by transfection with a nucleic acid construct encoding NLGN3 that may further encode a second label such as GFP). It is to be understood, that the first label and second label are conveniently and advantageously distinct (non-equivalent) labels, such as non-overlapping fluorophores, readily facilitating the evaluation or separation of EV bound by said affinity molecules by suitable methods such as fluorescent plate readers or flow cytometry.

In other embodiments, the positive control EV are obtained from cells (e.g. pluripotent stem cells) that were experimentally induced to express the one or more target molecules selected as isolation markers (such as synaptic protein targets as disclosed herein). For example, without limitation, the positive control EV may be obtained from induced pluripotent stem cells (IPS) differentiated ex vivo using e.g. suitable growth factors, hormones and/or media supplements, so as to express NDE-specific markers (e.g. NLGN3 as the isolation target and tau or p-Tau as the biomarker or diagnostic marker). For example, without limitation, growth factors and hormones including, but not limited to insulin, T3, retinoic acid, FGF2 and combinations thereof, or commercially available media such as DMEM or F12 supplemented by e.g. B27, may be used. In another exemplary embodiment, the positive control EV are obtained from disease-derived IPS (obtained from cells of a subject with the neurological condition of interest and differentiated ex vivo) and the negative control EV are obtained from IPS of a healthy subject (which are similarly differentiated ex vivo).

Non-EV Particles Control

In other embodiments, the methods of the invention further comprise supplementing the sample (or a separate aliquot of said sample) with a predetermined amount of labeled particles (e.g. bound to or containing a fluorophore) that display the same protein target (or at least one of the targets) used to capture the EV. For example, without limitation, the sample or aliquot may be combined with a control sample containing microparticles or other beads bound to a marker (e.g. a GAP43 polypeptide). According to these embodiments, the methods comprise determining the recovery rate of the beads or particles by quantifying the amount of labeled particles captured (e.g. by measuring fluorescent emission). It is to be understood, that quantifying the level of captured particles may also be performed by quantification of the uncaptured particles, and calculating the amount of captured particles accordingly.

The term “labeled beads” as used herein in connection to the methods and assays of the invention, relates to synthetic particles that are typically of a micrometer or nanometer scale, that are bound, directly or indirectly (e.g. via streptavidin-biotin interaction) to a target molecule as disclosed herein, and comprise a quantifiable label (e.g. fluorophore or dye) or that otherwise exhibit photoluminescent or fluorescent properties. Labeled beads are typically non-magnetic, and are distinct from beads that may be used as a substance for binding affinity molecules in EV isolation systems in their physical and chemical properties. It is to be understood, that when magnetic beads are used as a substrate for affinity molecules, labeled beads used as a quality control are not magnetic.

Suitable microparticle beads include, for example, melamine resin microspheres or polystyrene core beads. The quantifiable label may be e.g. a strong fluorophore or another type of detection signal, enabling the detection of low amounts of beads, and which does not interfere with the isolation of NDE. The beads are typically blocked in a similar manner to the substrate beads (used to bind the affinity molecules), to reduce non-specific interactions.

In other embodiments, the beads are nanoparticles, including, but not limited to, quantum dots. Quantum dots (QD) are semiconductor particles a few nanometers (nm) in size, having optical and electronic properties that differ from larger particles due to quantum mechanics. When the quantum dots are illuminated by UV light, an electron in the quantum dot can be excited to a state of higher energy. The excited electron can drop back into the valence band releasing its energy by the emission of light (photoluminescence). Their optoelectronic properties change as a function of both size and shape. For example, larger QD of 5-6 nm diameter generally emit longer wavelengths, with colors such as orange or red, whereas smaller QD (2-3 nm) generally emit shorter wavelengths, yielding colors like blue and green. The specific colors vary depending on the exact composition of the QD. Dots may be synthesized by e.g. colloidal synthesis, self-assembly and electrical gating, from materials including, but not limited to lead sulfide, lead selenide and cadmium selenide (in colloidal methods), or from silicon or germanium (plasma synthesis). For example, without limitation, Qdot Streptavidin Conjugate nanoparticles or similar products may be used. These QD are 15-20 nm in size and may provide emission in various colors (e.g. emission maxima of 525, 565, 585, 605, 655, or 705 nm).

As used herein, including the claims, the term “fluorophore” means a molecule with a functional group which can absorb energy of a specific wavelength and as a result emit energy at a different specific wavelength (i.e., a fluorescent molecule). Exemplary fluorophores include, but are not limited to, fluoresceins, rhodamines, coumarins, phthalocyanines, porphyrins, pyrenes, cyanines, squaraines, and boron-dipyrromethenes.

Synthetic and Recombinant Methods

Polypeptides and peptides to be used in the methods and assays of the invention (e.g. as isolation targets to be presented on particles, or polypeptide ligands of synaptic proteins), may be isolated or synthesized using any recombinant or synthetic method known in the art.

Synthetic methods include, without limitation, solid phase (e.g. Boc or f-Moc chemistry) and solution phase synthesis methods. For example, peptides can be synthesized by a solid phase peptide synthesis method of Merrifield (1963 J. Amer. Chem. Soc. 85:2149-2156). Alternatively, a peptide can be synthesized using standard solution methods well known in the art (see, for example, Bodanszky, 1984 The Principles of Peptide Synthesis, Springer-Verlag, New York) or by any other method known in the art for peptide synthesis. Peptides intended to be used as isolation targets may be chemically conjugated to control particles as disclosed herein, such as labeled beads.

In alternate embodiments, polypeptides and peptides may be produced by recombinant technology. Recombinant methods for designing, expressing and purifying proteins and peptides are known in the art (see, e.g. Sambrook et al., Molecular Cloning: A Laboratory Manual, Cold Springs Harbor Laboratory, New York, 2001). Nucleic acid molecules according to the invention may include DNA, RNA, or derivatives of either DNA or RNA. An isolated nucleic acid sequence encoding a polypeptide or peptide can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, natural allelic variants and modified nucleic acid sequences in which nucleotides have been inserted, deleted, substituted, and/or inverted in such a manner that such modifications do not substantially interfere with the nucleic acid molecule's ability to encode a functional polypeptide or peptide in accordance with the invention. A polynucleotide or oligonucleotide sequence can be deduced from the genetic code of a protein, however, the degeneracy of the code must be taken into account, as well as the allowance of exceptions to classical base pairing in the third position of the codon, as given by the so-called “Wobble rules”. For example, positive control EV engineered to express the one or more of the target molecules exogenously may be produced by expressing the target molecules on the surface of appropriate cells in culture according to recombinant technology, and collecting the resulting target-expressing EV.

Assay Parameters

According to exemplary embodiments, a recovery rate (e.g. of positive control target-specific EV or particles as disclosed herein) of at least 40% and typically at least 44%, 45%, 48% 50%, 54% or 55% indicates that the method or system is suitable for specific NDE isolation. In some embodiments, the methods of the invention are characterized by a recovery rate of at least 40% and typically at least 44%, 45%, 48% 50%, 54% or 55% of target-specific control particles. In a particular embodiment, a predetermined threshold corresponding to recovery of at least 44% of the predetermined amounts of said particles (that display the one or more of said target molecules, provided) in the control sample is applied.

The term “recovery rate” as used herein with respect to control EV or particles refers to the relative amount of bound particles, measured following contacting a sample, comprising a predetermined amount of the EV or particles, with substance-bound affinity molecules, using the methods or system to be evaluated. For determination of the recovery rate, the contacting is performed under conditions enabling specific binding of said affinity molecules to their corresponding target molecules (that are intended to be used in the method or system).

In some embodiments, the methods of the invention are characterized by sensitivity (evaluated by recovery of positive control EV) of at least eightfold or 10, 15, 20 or 50-fold higher compared to the respective recovery rates of EV isolated using a non-specific (Ig control) antibody. Additionally or alternatively, the methods of the invention are characterized by high specificity (e.g. evaluated by recovery of negative control EV which is at least 400-fold or 500, 600 or 800-fold lower, compared to the respective recovery rates of EV isolated using a non-specific antibody). In other embodiments, the methods of the invention are characterized by a ratio of the level of neuron-specific markers to the level of non-neuron-specific markers (e.g. platelet-specific markers) of at least tenfold and typically at least 20, 30, 40, 60, 80, 100 or 120-fold, and up to about 500-fold, in the isolated EV.

As disclosed and exemplified herein, the assays and methods of the invention exhibit remarkably improved accuracy as compared to hitherto reported NDE isolation and characterization methods. In particular, the assays and method of the invention provide for enhanced specificity compared to known methods using anti-L1CAM antibodies as sole affinity molecules for EV isolation. In particular, improved specificity of at least four-fold and typically at least 5, 8, 10, 12, 15, 20, 23, 25, 27, 30, 33, 36, 39, 42, 45, 46, 47 or 50-fold compared to the use of these hitherto known methods has been demonstrated in connection with the methods of the invention compared to the previous methods. For instance, Example 7 herein demonstrates improvement of the ratio of neuron-specific markers to platelet-specific markers of about 47-folds. In another embodiment, the methods and assays of the invention provide a robust platform for various clinical and research applications, including, but not limited to identification of new disease-associated markets and high throughput population screening. In another embodiment, the methods of the invention provide or are characterized by a Pearson correlation coefficient of at least 0.5 between the levels of neuron specific markers (e.g. proteins or RNA) in the isolated NDE and their levels in the respective brain tissue.

D. Applications and Diagnostic Assays

In various embodiments, the principles of the invention provide for improved diagnostic and analytical assays for various neurological diseases and conditions. In some embodiments, the methods and assays of the invention are useful for analyzing biological samples obtained from subjects afflicted with, suspected of having, or considered at risk for developing, the neurological condition. In various embodiments, the methods and assays are used for diagnosing, assessing the risk of developing, monitoring the progression of, prognosing, or providing differential diagnosis of neurological and neurodegenerative conditions and pathologies. The biomarkers measured from the isolated EV, and advantageously also the isolation targets and corresponding affinity molecules, may be selected accordingly, as disclosed herein.

Indications

In some embodiments, a sample to be used in connection with the assays, methods and systems of the invention is obtained from a subject afflicted with, or suspected of having or being predisposed to, a neurodegenerative disease. As used herein, a “neurodegenerative disease” refers to a disease in which degeneration occurs in either gray or white matter, or both, of the nervous system. For example, without limitation, the sample may be obtained from a subject afflicted with, or suspected of having or being predisposed to Mild Cognitive Impairment (MCI), Alzheimer's Disease (AD), or dementia due to other causes (including, but not limited to, frontotemporal dementia), wherein each possibility represents a separate embodiment of the invention.

In other embodiments, the sample may be taken from a subject afflicted with, or suspected of having or being predisposed to, a neurological (including neurodegenerative) condition including, but not limited to, corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), multiple sclerosis and traumatic brain injury (TBI), wherein each possibility represents a separate embodiment of the invention.

Alzheimer's Disease (AD) is a neurocognitive disorder, characterized by β-amyloid deposits and neurofibrillary tangles in the cerebral cortex and subcortical gray matter. AD is the most common cause of dementia and accounts for 60 to 80% of dementias in older people. The percentage of people with AD increases with age and is twice as common among women compared to men. Mutations in genes for the amyloid precursor protein (APP), as well as in presenilin-1 and presenilin-2 (PSEN1 and PSEN2, respectively), may lead to autosomal dominant forms of AD, typically with presenile onset (familial AD). Other genetic determinants include apolipoprotein (apo) E (epsilon) alleles which influence β-amyloid deposition, and TREM2 mutations which affect neuroinflammation. Vascular risk factors (e.g. smoking or other chemical inducers) can increase the risk of AD. The most common first manifestation of AD is loss of short-term memory. Other cognitive deficits tend to involve multiple functions, including impaired reasoning, difficulty handling complex tasks, and poor judgment, language dysfunction, and visuospatial dysfunction. Additional manifestations of AD are behavior disorders such as, wandering, agitation, yelling, and persecutory ideation. Treatment of AD includes safety and support measures, and may also include cholinesterase inhibitors and memantine.

In some embodiments, a sample to be used in connection with the assays, methods and systems of the invention is obtained from a subject suspected of having AD or of being predisposed to develop AD.

In some embodiments, the subject is suspected of having AD. Typically, the subject is suspected of having AD based on the presence of one or more symptoms or disease manifestations of AD, e.g. as specified hereinabove. In certain embodiments, the subject is presented with loss of short-term memory, impaired reasoning, difficulty handling complex tasks, poor judgment, language dysfunction, and/or visuospatial dysfunction. In particular, a subject may be suspected of having AD based on at least the presence of dementia. For example, as exemplified herein, a diagnostic signature based on altered levels of Aβ42, p-Tau, PSD95 and proBDNF gene products, was identified in AD patients exhibiting signs of dementia. Thus, according to exemplary embodiments of the methods of the invention, the sample is obtained from a subject exhibiting signs of dementia and suspected of having AD, and the method comprises determining the levels of Aβ42, p-Tau, PSD95 and proBDNF.

Additionally or alternatively, the subject may be suspected of having AD, or of being predisposed to developing AD, based on the presence of one or more risk factors associated with AD. As used herein, “predisposed” to a disease such as a neurodegenerative disease may refer to a genetic, familial or chemically-induced predisposition, e.g. as described and exemplified herein. A subject suspected of being predisposed to a disease may be identified as a suspect thereto based on one or more of the above risk factors, even in the absence of any symptoms or disease manifestations.

In some embodiments, the subject has a genetic mutation known to be correlated with a risk of developing AD. In some embodiments, the subject is carrying the F4 or 3 allele of Apo E. In other embodiments, the subject has a mutation in at least one gene selected from the group consisting of amyloid precursor protein gene (APP), presenilin 1 gene (PSEN1) and presenilin 2 gene (PSEN2).

A subject determined to be predisposed (or diagnosed as being predisposed) to a disease using the methods of the invention, is confirmed and considered to be highly likely to develop disease symptoms within several years (e.g. 1-3) of the diagnosis. Such subjects are typically asymptomatic or minimally symptomatic (or in some cases—present with symptoms that are not specific or unique to the disease), but are characterized by an active pathological process that underlies disease development. Without wishing to be bound by a specific theory or mechanism of action, such pathological processes may now be identified at an early stage with exceptional accuracy. This determination or diagnosis of predisposition in the subject may be used to assist a treating physician to determine a treatment or management plan for the subject. For example, in some embodiments an early onset of prophylactic treatment for a neurodegenerative disease may be indicated in e.g. pre-symptomatic subjects determined to be predisposed to developing the disease in accordance with the methods and assays of the invention.

In other embodiments, the identification of a subject suspected of being predisposed to a disease may be done based on the presence of another disease or condition that is considered to be a risk factor, contributor or a predictor to the development of the disease. For example, in some cases, MCI patients can eventually develop AD, as will be explained in further detail below.

In some embodiments, the methods of the invention comprise a step of providing or assigning a therapy to a subject that had been diagnosed with AD in accordance with the methods and assays of the invention (e.g. using a diagnostic signature for AD as exemplified herein). In some embodiments, the methods of the invention comprise a step of treating AD. In some embodiments, the methods of the invention comprise a step of administering to the subject diagnosed with AD in accordance with the methods of the invention, a therapy selected from the group consisting of a cholinesterase inhibitor, memantine, and aducanumab. According to some embodiments, the cholinesterase inhibitor is selected from the group consisting of donepezil, rivastigmine, galantamine and tacrine. Each possibility represents a separate embodiment of the invention.

Mild Cognitive Impairment (MCI) is a neurological syndrome defined as cognitive decline greater than expected for an individual's age and education level but that does not interfere notably with activities of daily life. MCI was recognized as a clinical entity in the 1980's and the clinical criteria for diagnosing MCI were described by Dr. Ronald C. Petersen et al in 1999 and included: (1) memory complaint, (2) normal activities of daily living, (3) other cognitive functions normal, (4) abnormal memory for age, and (5) no dementia. These criteria were widely adopted with refinements based on subsequent Consensus Conferences and are being used by clinicians for diagnosing the majority of MCI cases. The evaluation of cognitive complaints requires the use of neuropsychological and cognitive assessment batteries.

Thus, this condition is distinguishable from other conditions such as age-associated memory impairment (in which the impairment is characteristic of aging), and dementia (in which the impairment is characterized in that daily functioning is impaired). Other neurological or mental disorders affecting cognitive function, such as AD and other neurodegenerative conditions (e.g. FTD, Lewy body dementia), chronic traumatic encephalopathy, traumatic brain injury, stroke, brain tumors, subdural hematoma, normal pressure hydrocephalus, depression, as well as metabolic, endocrine, infectious and toxic conditions, should be excluded prior to diagnosing a person with MCI, in order to accurately diagnose MCI. Recent research has shown that a number of individuals with MCI have an increased risk of developing AD. However, the conversion rate from MCI to AD is small (typically about 10-15%), and consequently a diagnosis of MCI does not mean that the person will eventually develop AD.

The treatment of MCI patients commonly includes non-pharmacological interventions such as physical exercise, cognitive training and dietary advice. In addition, several pharmacological agents, including, but not limited to, sodium benzoate, serotonergic agents or other agents stimulating soluble amyloid precursor protein secretion, and low doses of certain cholinesterase inhibitors, have been suggested for the management of MCI.

In some embodiments, a sample to be used in connection with the assays, methods and systems of the invention is obtained from a subject suspected of having MCI. A subject suspected of having MCI typically manifests memory loss, that may be recognized initially either as self-reported complaints or evaluated by the physician using e.g. standardized cognitive tests).

Dementia is a syndrome characterized by chronic, global, usually irreversible deterioration of cognition. Dementia may occur at any age but affects primarily older people. Dementia may be differentiated from other conditions such as delirium in that memory is typically impaired (rather than mainly attention in delirium) and that the impairment is chronic and typically permanent, and form conditions such as MCI in that the impairment is severe enough to interfere with daily living. This syndrome may be manifested in patients afflicted with certain neurodegenerative diseases including, but not limited to AD, FTD, vascular dementia, Lewy body dementia, Limbic-predominant age-related TDP-43 encephalopathy (LATE) and tangle-predominant senile dementia.

Dementia can be divided into an early, intermediate or late dementia based on the symptoms. Cognitive changes may be conveniently evaluated by standardized cognitive tests (e.g. Mini-Mental Status Examination (MMSE), and the Montreal Cognitive Assessment (MoCA) scale). Personality changes and behavioral disturbances may develop early or late. Motor and other focal neurologic deficits occur at different stages, depending on the type of dementia; they occur early in vascular dementia and late in dementia associated with AD. Incidence of seizures is somewhat increased during all stages.

Frontotemporal dementia (FTD) refers to sporadic and hereditary disorders that affect the frontal and temporal lobes. These conditions are associated with brain accumulation of different proteins (proteinopathies, including mainly Tau and Transactive response DNA binding protein of 43 kDa (TDP43). About half of FTD cases are inherited; many known mutations involve chromosome 17q21-22 and result in abnormalities of the microtubule-binding tau protein. However, to date, the proteinopathies can only be suggested based on the type of symptoms, in a highly error-prone manner, and accurate diagnosis can only be done postmortem.

Generally, FTD affects personality, behavior, and usually language function (syntax and fluency). Abstract thinking and attention (maintaining and shifting) are impaired; responses are disorganized. Orientation is preserved, but retrieval of information may be impaired. Motor skills are generally preserved. Patients may have difficulty sequencing tasks, although visuospatial and constructional tasks are affected less. Some patients develop motor neuron disease with generalized muscle atrophy, weakness, fasciculations, bulbar symptoms (e.g. dysphagia, dysphonia, difficulty chewing), and increased risk of aspiration pneumonia and early death. Semantic dementia is a type of primary progressive aphasia. When the left side of the brain is affected most, the ability to comprehend words is progressively lost. Speech is fluent but lacks meaning; a generic or related term may be used instead of the specific name of an object. When the right side is affected most, patients have progressive anomia (inability to name objects) and prosopagnosia (inability to recognize familiar faces). They cannot remember topographic relationships.

There are different types of FTD, depending on which part of the brain is affected. In behavioral (frontal) variant FTD, social behavior and personality change because the orbitobasal frontal lobe is affected. In primary progressive aphasia, language function deteriorates because of asymmetric (worse on left) anterolateral temporal lobe atrophy; the hippocampus and memory are relatively spared. Pick disease is a term used to describe pathologic changes in FTD, including severe atrophy, neuronal loss, gliosis, and presence of abnormal neurons (Pick cells) containing inclusions (Pick bodies).

The diagnosis of FTD is similar to dementia but include additional clinical evaluation to differentiate from some other dementias. CT and MRI may be done to determine location and extent of brain atrophy and to exclude other possible causes (e.g. brain tumors, abscesses, stroke). FTDs are characterized by severely atrophic, sometimes paper-thin gyri in the temporal and frontal lobes. However, MRI or CT may not show these changes until late in FTD.

In some embodiments, a sample to be used in connection with the assays, methods and systems of the invention is obtained from a subject suspected of having FTD (e.g. due to symptoms or manifestations as disclosed herein). In other embodiments, a sample to be used in connection with the assays, methods and systems of the invention is obtained from a subject diagnosed with FTD (e.g. as disclosed herein). in some embodiments, the methods described herein can distinguish between FTD pathologies (e.g. proteinopathies as disclosed herein), enabling for the development or selection of personalized medicines or therapies.

In another particular embodiment, the condition is ALS. ALS is the most common motor neuron disease. ALS is characterized by steady, relentless, progressive degeneration of corticospinal tracts, anterior horn cells, and/or bulbar motor nuclei. Symptoms vary in severity and may include muscle weakness and atrophy, fasciculations, emotional lability, and respiratory muscle weakness. Other symptoms include hoarseness, dysphagia, and slurred speech; because swallowing is difficult, salivation appears to increase, and patients tend to choke on liquids. Late in the disorder, a pseudobulbar affect occurs, with inappropriate, involuntary, and uncontrollable excesses of laughter or crying. Sensory systems, consciousness, cognition, voluntary eye movements, sexual function, and urinary and anal sphincters are usually spared. Death is usually caused by failure of the respiratory muscles; 50% of patients die within 3 years of onset, and survival for >30 years is rare.

Diagnosis of ALS usually involves electrodiagnostic tests and MRI of the brain and, if no cranial nerve involvement, cervical spine. Additional diagnosis is needed to rule out other disorders that cause pure muscle weakness, e.g., disorders of neuromuscular transmission, various myopathies (including noninflammatory and drug-induced), spinal muscular atrophies (mostly in children), polymyositis, and dermatomyositis. This process is lengthy and cumbersome, and requires about 14 months on average for a confirmed diagnosis according to hitherto known methods. In addition, many patients are initially misdiagnosed and thus undergo unnecessary orthopedic procedures during the time period required to obtain a correct diagnosis.

Treatments of ALS include mainly supportive care, and medicines such as, Riluzole (that may provide improvement in survival by 2 to 3 months) and Edaravone (that may slow the decline in function to some degree). Additional drugs that may help reduce symptoms include baclofen (against spasticity), quinine or phenytoin (against cramps), anticholinergic drug (to decrease saliva production), and amitriptyline, fluvoxamine, or a combination of dextromethorphan and quinidine to prevent pseudobulbar affects.

In some embodiments, a sample to be used in connection with the assays, methods and systems of the invention is obtained from a subject suspected of having ALS. In some embodiments, the subject is suspected of having ALS based on the presence of one or more symptoms or disease manifestations of ALS, e.g. as specified hereinabove. In certain embodiments, the subject is presented with an ALS symptom selected from the group consisting of muscle weakness and atrophy, fasciculations, emotional lability, respiratory muscle weakness, and any combination thereof.

In some embodiments, the methods of the invention comprise providing or assigning a therapy to a subject that had been diagnosed with ALS according to a method as disclosed herein. In some embodiments, the methods of the invention comprise a step of treating ALS. In some embodiments, the methods of the invention comprise a step of administering to the subject diagnosed with ALS (e.g. using a diagnostic signature for ALS as exemplified herein), a therapy selected from the group consisting of baclofen, quinine or phenytoin, anticholinergic drug, amitriptyline, fluvoxamine, a combination of dextromethorphan and quinidine, and any combination thereof.

Methods

In one aspect, there is provided a method of identifying or capturing neuron-derived EV, comprising:
a. providing an EV-containing biofluid sample,
b. contacting the sample with affinity molecules capable of binding to target molecules on the surface of the EV, wherein said target molecules comprise NLGN3 and GAP43, under conditions enabling specific binding of the affinity molecules to their corresponding target molecules, and
c. identifying or capturing the EV bound to the affinity molecules.

In another embodiment, said target molecules are NLGN3 and GAP43. In another embodiment, said target molecules further comprise L1CAM. In yet another embodiment, said target molecules are NLGN3, GAP43 and L1CAM. In one embodiment, said affinity molecules are substance-bound. In a particular embodiment, the substance is a plurality of magnetic beads.

In another embodiment, the method further comprises:
i. providing a control sample containing predetermined amounts of particles that display the target molecules (or at least one of said target molecules),
ii. contacting the control sample with the affinity molecules, under the conditions enabling specific binding to their corresponding target molecules,
iii. quantifying the amount of particles bound by said affinity molecules, and
iv. determining that said amount is over a predetermined threshold.

In another embodiment, the particles are labeled, e.g. fluorescently. In another embodiment, said particles are beads (e.g. microparticles). In a particular embodiment said beads are labeled by a dye, e.g. a fluorophore. In another embodiment, the particles are fluorescently labeled beads, added to (combined with) the biofluid sample prior to contacting said biofluid sample with said affinity molecules. In another embodiment, the particles are control EV obtained from engineered cells. In another embodiment the control EV comprise positive control EV engineered to express the target molecules exogenously. In another embodiment the control EV comprise negative control EV that do not express the target molecules. In a particular embodiment, the control EV comprise negative control EV obtained from non-neuronal cells that contain a first fluorescent label, and positive control EV obtained from the same (or equivalent) non-neuronal cells engineered to express the target molecules, and a second, distinct fluorescent label.

In another embodiment, the method further comprises determining the levels of one or more biomarkers in the EV bound to the affinity molecules. In various embodiments, the biomarkers are selected from the group consisting of protein, nucleic acid, lipid and/or metabolite biomarkers. According to exemplary embodiments, the biomarkers are selected from the group consisting of Tau (total protein or phosphorylated fraction), Amyloid-beta 42 (Aβ42), NLGN3, TDP43, clusterin, SYP, BIM, NEFL, ENO2, NRGN, and GPR26 gene products, and combinations thereof. In various other embodiments, the biomarkers are selected from the group consisting of Tau, p-Tau, Aβ42, and NLGN, TDP43, CLU, SYP, BIM, NEFL, ENO2, NRGN, Cathepsin D, LC3, SYT and GPR26 gene products, and combinations thereof. In another embodiment the one or more biomarkers are selected from the group consisting of: LC3, Cathepsin D, NRF2, Aβ42, p-Tau, PSD95, proBDNF, COX2, EIF2C2 and NF-κB. In another embodiment said biomarkers are Aβ42, p-Tau, PSD95 and proBDNF. In another embodiment said biomarkers are LC3, TDP43, and NRF2 Each possibility represents a separate embodiment of the invention.

In another embodiment the sample is obtained from a subject suspected of having MCI (including MCI associated with AD), or of being predisposed to developing AD, and the biomarkers are total-Tau, p-Tau (including, in particular, p181-Tau), Aβ42, and Neuroligin (NLGN, including in particular NLGN1). In another embodiment, the method further comprises comparing the levels of the biomarkers in the bound EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with MCI.

In various embodiments of the methods of the invention, a control biofluid sample is selected from the group consisting of a sample from at least one healthy individual, a panel of control samples from a set of healthy individuals, and a stored set of data obtained from healthy individuals. Each possibility represents a separate embodiment of the invention. In certain other embodiments (such as when the methods are used for differentiating between diseases or pathologies), the control sample may be selected from the group consisting of a sample from at least one diagnosed individual (e.g. with a known FTD pathology), a panel of control samples from a set of similarly-diagnosed individuals, and a stored set of data obtained from similarly-diagnosed individuals. Each possibility represents a separate embodiment of the invention.

In another embodiment the sample is obtained from a subject diagnosed with, or suspected of having, FTD, and the biomarkers are TDP43 and p-Tau. In another embodiment, the method further comprises comparing the levels of the one or more biomarkers in the bound EV to their respective levels corresponding to control biofluid samples, and characterizing the FTD pathology in said subject based on the levels of TDP43 and p-Tau determined in the isolated EV.

In another embodiment, the sample is obtained from a subject suspected of having AD, and said biomarkers are Aβ42, p-Tau, PSD95 and proBDNF. In a particular embodiment, the subject is suspected of having AD based on at least the presence of dementia. In another embodiment, the sample is obtained from a subject exhibiting dementia and suspected of having AD, and said biomarkers are Aβ42, p-Tau, PSD95 and proBDNF. In another embodiment, the method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with AD.

In another embodiment, the sample is obtained from a subject suspected of having ALS, and said biomarkers are LC3, TDP43, and NRF2. In another embodiment, the method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with ALS.

In another aspect, there is provided a method for isolating neuron-derived EV, comprising:
a. providing an isolation system comprising substance-bound affinity molecules capable of binding to one or more target molecules on the surface of the EV, wherein at least one target molecule is a synaptic protein,
b. providing a control sample containing predetermined amounts of particles that display the one or more target molecules (or at least one of said target molecules),
c. determining the accuracy of the system, by:
   i. contacting said system with the control sample, under conditions enabling specific binding of said affinity molecules to their corresponding target molecules,
   ii. quantifying the amount of particles bound by said affinity molecules, and
   iii. determining that said amount is over a predetermined threshold,
d. providing an EV-containing biofluid sample,
e. contacting the biofluid sample with said system, under the conditions enabling specific binding of said affinity molecules to their corresponding target molecules, and
f. isolating the EV bound to said affinity molecules.

In another embodiment, said synaptic protein is selected from the group consisting of NLGN3, GAP43, SYT1, and GRIA1. In another embodiment, said target molecules comprise NLGN3 and GAP43. In another embodiment, said target molecules are NLGN3 and GAP43. In another embodiment, said target molecules further comprise L1CAM and/or Rab3a. In yet another embodiment, said target molecules are NLGN3, GAP43 and L1CAM. In one embodiment, said affinity molecules are substance-bound. In a particular embodiment, the substance is a plurality of magnetic beads (typically superparamagnetic microparticles). For example, without limitation, the invention may employ the use of various commercially available magnetic bead isolation systems including, but not limited to, BioMag, Dynabeads, MyOne or Mag Sepharose. These systems typically utilize superparamagnetic microparticles (beads) of 1-3 μm comprised of iron (e.g. iron oxide) and optionally various polymers. The surface of these particles may be coated with various functional groups or elements (e.g. streptavidin or the like) enabling conjugation of the antibodies or other affinity molecules. The systems further utilize a magnet which is conveniently adapted for use with a tube rack.

In another embodiment, the particles of the control sample are labeled by a marker, e.g. a fluorophore. In another embodiment, said particles are beads. In a particular embodiment said beads are labeled by a dye, e.g. a fluorophore. In another embodiment, the particles are fluorescently labeled beads, and the control sample is added to the biofluid sample prior to contacting said biofluid sample with said affinity molecules. In another embodiment, the particles are control EV obtained from engineered cells. In another embodiment the control EV comprise positive control EV engineered to express the target molecules exogenously. In another embodiment the control EV comprise negative control EV that do not express the target molecules. In a particular embodiment, the control EV comprise negative control EV obtained from non-neuronal cells that contain a first fluorescent marker, and positive control EV obtained from equivalent non-neuronal cells engineered to express the target molecules, and a second, distinct fluorescent marker. In various embodiments, step c is performed prior to, concomitantly with, or after step d. For example, for an internal, spike-in control, the particles of the control sample are combined with the EV-containing sample, and step c. is performed concomitantly with step d. In another example, the control sample is contacted with the system separately from the EV-containing sample (either before, after or simultaneously). Thus, it is understood that step c. may be performed at any point after step b., wherein each possibility represents a separate embodiment of the invention.

In another embodiment, the predetermined threshold corresponds to recovery of at least 44%, 45%, 48% 50%, 54% or 55% of the predetermined amount of particles provided in the control sample. In a particular embodiment the predetermined threshold corresponds to recovery of at least 44% of the predetermined amount of particles that display the one or more of said target molecules, provided in the control sample. In another embodiment, the isolated EV are characterized by a ratio of neuron-specific markers to non-neuron-specific markers (e.g. platelet-specific markers) of at least 20, 30, 40, 60, 80, 100 or 120-fold.

In another embodiment, the method further comprises determining the levels of one or more biomarkers in the EV bound to the affinity molecules (e.g. in the EV lysate or on the surface of said EV). In various embodiments, the biomarkers are selected from the group consisting of protein, nucleic acid, lipid and/or metabolite biomarkers. According to exemplary embodiments, the biomarkers are selected from the group consisting of Tau, phosphorylated Tau (p-Tau), Aβ42, NLGN3, TDP43, clusterin, SYP, BIM, NEFL, ENO2, NRGN, and GPR26, NRXN, BAD, and phosphorylated BAD (p-BAD) gene products, and combinations thereof. In various other embodiments, the biomarkers are selected from the group consisting of the one or more biomarkers are selected from the group consisting of Tau, p-Tau, Aβ42, and NLGN, TDP43, CLU, SYP, BIM, NEFL, ENO2, NRGN, Cathepsin D, LC3, NRF2, SYT and GPR26 gene products, and combinations thereof. Each possibility represents a separate embodiment of the invention.

In another embodiment the sample is obtained from a subject suspected of having MCI, or of being predisposed to developing AD, and the biomarkers are total-Tau, p-Tau, Aβ42, and NLGN gene products (e.g. proteins). In another embodiment, the method further comprises comparing the levels of the biomarkers in the bound EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with MCI.

In another embodiment the sample is obtained from a subject diagnosed with, or suspected of having, FTD, and the biomarkers are TDP43 and p-Tau gene products. In another embodiment, the method further comprises comparing the levels of the one or more biomarkers in the bound EV to their respective levels corresponding to control biofluid samples, and characterizing the FTD pathology in said subject based on the levels of TDP43 and p-Tau determined in the isolated EV.

In another embodiment, the sample is obtained from a subject suspected of having AD, and said biomarkers are Aβ42, p-Tau, PSD95 and proBDNF. In another embodiment, the method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with AD.

In another embodiment, the sample is obtained from a subject suspected of having ALS, and said biomarkers are LC3, TDP43, and NRF2. In another embodiment, the method further comprises comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with ALS.

In another aspect, there is provided a method for analyzing a biofluid sample, comprising determining the levels of one or more biomarkers in neuron-derived EV of the sample, wherein
a. the sample is obtained from a subject suspected of having MCI, or of being predisposed to developing AD, and the biomarkers are total-Tau, p-Tau, Aβ42, and NLGN gene products;
b. the sample is obtained from a subject diagnosed with, or suspected of having, FTD, and the biomarkers are TDP43 and p-Tau gene products;
c. the sample is obtained from a subject suspected of having AD, and the biomarkers are Aβ42, p-Tau, PSD95 and proBDNF; or
d. the sample is obtained from a subject suspected of having ALS, and the biomarkers are LC3, TDP43, and NRF2.

In various embodiments, analyzing a biofluid sample comprises detection and/or quantification of the EV payload (e.g. gene expression or biomarker levels, as disclosed herein).

In one embodiment, the method further comprises comparing the levels of the biomarkers in the isolated EV to their respective levels corresponding to a control biological sample. In another embodiment the method further comprises, prior to determining the levels of the biomarkers in the neuron-derived EV, a step of isolating said EV from the sample, by contacting said sample with surface-bound affinity molecules capable of binding to one or more target molecules on the surface of said EV, wherein at least one target molecule is a synaptic protein as disclosed herein, under the conditions enabling specific binding of said affinity molecules to their corresponding target molecules, and isolating the EV bound to the affinity molecules. In a particular embodiment said affinity molecules are directed to NLGN3 and GAP43 and the substance is a plurality of magnetic beads. In another embodiment the method further comprises:
i. providing a control sample containing predetermined amounts of particles that display the target molecules (or at least one of said target molecules),
ii. contacting the control sample with the affinity molecules, under the conditions enabling specific binding to their corresponding target molecules,
iii. quantifying the amount of particles bound by said affinity molecules, and
iv. determining that said amount is over a predetermined threshold.

In various embodiments, the method is used for diagnosing, assessing or predicting the development of a neurological or neurodegenerative condition, e.g. a disease or condition as disclosed herein. In another embodiment the method is used for diagnosing MCI in a subject in need thereof.

In another embodiment there is provided a method for diagnosing MCI in a subject in need thereof, comprising:
a. obtaining an EV-containing biofluid sample from the subject,
b. providing an isolation system comprising surface-bound affinity molecules capable of binding to one or more targets on the surface of the EV, wherein at least one target is a synaptic protein selected from the group consisting of NLGN3 and GAP43,
c. contacting the biofluid sample with said system, under conditions enabling specific binding of said affinity molecules to their corresponding targets,
d. isolating the EV bound to the affinity molecules,
e. determining the levels of total-Tau, p-Tau, Aβ42, and NLGN in the isolated EV, and
f. comparing the levels of the biomarkers in the isolated EV to their respective levels corresponding to a control biofluid sample, to thereby compare the diagnostic signature of the sample to the control diagnostic signature, wherein a significant difference in the diagnostic signature of the sample compared to the control diagnostic signature indicates that said subject is afflicted with MCI.

In another embodiment the method is used for diagnosing AD in a subject in need thereof. In another embodiment there is provided a method for diagnosing AD in a subject in need thereof, comprising:
a. obtaining an EV-containing biofluid sample from the subject,
b. providing an isolation system comprising surface-bound affinity molecules capable of binding to one or more targets on the surface of the EV, wherein at least one target is a synaptic protein selected from the group consisting of NLGN3 and GAP43,
c. contacting the biofluid sample with said system, under conditions enabling specific binding of said affinity molecules to their corresponding targets,
d. isolating the EV bound to the affinity molecules,
e. determining the levels of Aβ42, p-Tau, PSD95 and proBDNF in the isolated EV, and
f. comparing the levels of the biomarkers in the isolated EV to their respective levels corresponding to a control biofluid sample, to thereby compare the diagnostic signature of the sample to the control diagnostic signature,
   wherein a significant difference in the diagnostic signature of the sample compared to the control diagnostic signature indicates that said subject is afflicted with AD.

In another embodiment, the method is used for diagnosing AD or predisposition thereto in a subject in need thereof, and comprises:
a. obtaining an EV-containing biofluid sample from the subject,
b. providing an isolation system comprising substance-bound affinity molecules capable of binding to target molecules comprising NLGN3 and GAP43 on the surface of the EV,
c. contacting the biofluid sample with said system, under conditions enabling specific binding of said affinity molecules to their corresponding target molecules,
d. isolating the EV bound to said affinity molecules,
e. determining the levels of at least four biomarkers comprising at least one synaptic protein, at least one Tau gene product and at least one amyloid gene product in the isolated EV, and
f. comparing the levels of the biomarkers as determined in the isolated EV to their respective levels corresponding to a control biofluid sample, to thereby compare the diagnostic signature of the sample to the control diagnostic signature, wherein a significant difference in the diagnostic signature of the biofluid sample compared to the control diagnostic signature indicates that said subject is afflicted with, or predisposed to developing, AD.

In various embodiments, the synaptic protein biomarker is selected form PSD95, and NLGN1, the Tau gene product is selected from total-Tau and p-Tau (in particular p181-Tau), and the amyloid gene product is Aβ42. In another embodiment, an additional biomarker is proBDNF.

In another embodiment said biomarkers are Aβ42, p-Tau, PSD95 and proBDNF, and a significant difference in the diagnostic signature of the biofluid sample compared to the control diagnostic signature indicates that said subject is afflicted with AD.

In yet another embodiment, the method is used for characterizing an FTD pathology in a subject in need thereof. In another embodiment there is provided a method of characterizing an FTD pathology in a subject in need thereof, comprising:
a. obtaining an EV-containing biofluid sample from the subject,
b. providing an isolation system comprising surface-bound affinity molecules capable of binding to one or more targets on the surface of the EV, wherein at least one target is a synaptic protein selected from the group consisting of NLGN3 and GAP43,
c. contacting the biofluid sample with said system, under conditions enabling specific binding of said affinity molecules to their corresponding targets,
d. isolating the EV bound to the affinity molecules,
a. determining the levels of TDP43 and p-Tau in the isolated EV, and
b. comparing the levels of the one or more biomarkers in the isolated EV to their respective levels corresponding to control biofluid samples, and characterizing the FTD pathology in said subject based on the levels of TDP43 and p-Tau determined in the isolated EV.

In another embodiment, characterizing the FTD pathology includes determining whether the sample is characterized by enhanced levels of TDP43 and reduced levels of p-Tau, and/or determining whether the sample is characterized by reduced levels of TDP43 and enhanced levels of p-Tau. In another embodiment, the control biofluid sample corresponds to a pool of samples from FTD patients.

In another embodiment, enhanced levels of TDP43 and reduced levels of p-Tau in the isolated EV compared to control levels corresponding to a pool of FTD patients indicates that said FTD pathology is associated with a TDP43 proteinopathy. In another embodiment, reduced levels of TDP43 and enhanced levels of p-Tau in the isolated EV compared to control levels corresponding to a pool of FTD patients indicates that said FTD pathology is associated with a Tau proteinopathy. In yet another embodiment, enhanced levels of TDP43 and reduced levels of p-Tau in the isolated EV compared to control levels corresponding to a pool of FTD patients indicates that said FTD pathology is associated with a TDP43 proteinopathy, and reduced levels of TDP43 and enhanced levels of p-Tau in the isolated EV compared to control levels corresponding to a pool of FTD patients indicates that said FTD pathology is associated with a Tau proteinopathy.

As used herein, enhanced or reduced levels of a biomarker as disclosed herein (e.g. TDP43 or p-Tau), refers to a significant difference (elevation or reduction, respectively). A “significant difference”, when used in connection with the methods of the invention refers to a statistically significant difference and/or to a significant difference as evaluated by the skilled artisan (e.g. the treating physician) according to the art-recognized acceptable criteria.

In a further embodiment, the method is used for diagnosing ALS in a subject in need thereof. In another embodiment there is provided a method for diagnosing ALS in a subject in need thereof, comprising:
a. obtaining an EV-containing biofluid sample from the subject,
b. providing an isolation system comprising surface-bound affinity molecules capable of binding to one or more targets on the surface of the EV, wherein at least one target is a synaptic protein selected from the group consisting of NLGN3 and GAP43,
c. contacting the biofluid sample with said system, under conditions enabling specific binding of said affinity molecules to their corresponding targets,
d. isolating the EV bound to the affinity molecules,
e. determining the levels of LC3, TDP43, and NRF2 in the isolated EV, and
f. comparing the levels of the biomarkers in the isolated EV to their respective levels corresponding to a control biofluid sample, to thereby compare the diagnostic signature of the sample to the control diagnostic signature,
   wherein a significant difference in the diagnostic signature of the sample compared to the control diagnostic signature indicates that said subject is afflicted with ALS.

According to embodiments of the methods of the invention, the affinity molecule is an antibody or an antigen-binding portion thereof. In additional embodiments of the methods of the invention, the biofluid or biological sample is selected from the group consisting of blood, plasma and serum. In other embodiments, the subject is human. In a particular embodiment, the sample is obtained from a human subject suspected or considered predisposed to develop the disease. In yet another embodiment, the sample is obtained from a human subject that is being tested as part of routine or scheduled screening for the disease. For example, without limitation, subjects over the age of 50, 55, 60, 65 or more may be screened to identify their likelihood to develop AD, e.g. within 5 years of testing, by methods as disclosed herein, involving e.g. determining the levels of total-Tau, p-Tau, Aβ42, and NLGN in the isolated EV. In other embodiments, the sample may be obtained from mice, rats or other mammals useful e.g. in pre-clinical studies. Each possibility represents a separate embodiment of the invention.

Data Analysis

According to embodiments of the invention, substantial difference or similarity of diagnostic signatures are determined considering the collective levels of the biomarkers (e.g. gene products) of the signature. In some embodiments, a substantially different diagnostic signature compared to a control comprises significantly enhanced levels of a set of gene products as disclosed herein compared to their respective control levels. In other embodiments a substantially different diagnostic signature compared to a control comprises significantly reduced levels of a set of gene products as disclosed herein compared to their respective control levels. In yet other embodiments, a substantially different diagnostic signature compared to a control comprises both significantly enhanced levels of one or more markers as disclosed herein and significantly reduced levels of one or more additional markers as disclosed herein compared to their respective control levels. Each possibility represents a separate embodiment of the invention.

Advantageously, the methods of the invention can employ the use of learning and pattern recognition analyzers, clustering algorithms and the like, in order to discriminate between the diagnostic signature of a sample or subject and control diagnostic signatures as disclosed herein. For example, the methods can comprise determining the levels of biomarkers as disclosed herein in EV isolated from a biofluid sample, and comparing the resulting diagnostic signature to a control diagnostic signature using such algorithms and/or analyzers.

In certain embodiments, one or more algorithms or computer programs may be used for comparing the amount of each gene product quantified in the sample against a predetermined cutoff (or against a number of predetermined cutoffs). Alternatively, one or more instructions for manually performing the necessary steps by a human can be provided.

In some embodiments, receiver operating characteristics (ROC) analysis and AUC plus probabilistic metrics (e.g., log-loss) may be used in connection with the methods of the invention. Hypothesis-based signature development, where pre-knowledge on the biomarker role in the disease may be taken under consideration. In other embodiments, linear mixed-effect algorithms may be used to model differences in selected biomarker(s). In other embodiments, machine learning (ML) is used to evaluate the biomarkers as potential indicators of progression, exploiting temporal heterogeneous effects, as well as sparse and varying-length patient characteristics commonly seen with disease progression. Mixed-effect machine learning and long- and short-term memory (LS™) neural networks may be used to predict changes in biomarker trajectories and to classify patients. Multivariate methods (e.g., logistic regression, K-nearest neighbor, support vector machine, and machine learning) can also be used. A class of non-linear algorithms that show better performance in small and medium-sized datasets including decision tree-based methods (i.e., random forest, gradient boosting) and support vector machines may also be used. Algorithms for determining and comparing diagnostic signatures further include, but are not limited to, supervised classification algorithms including, but not limited to, gradient boosted trees, random forest, regularized regression, multiple linear regression (MLR), principal component regression (PCR), partial least squares (PLS), discriminant function analysis (DFA) including linear discriminant analysis (LDA), nearest neighbor, artificial neural networks, multi-layer perceptrons (MLP), generalized regression neural network (GRNN), and combinations thereof, or non-supervised clustering algorithms, including, but not limited to, K-means, spectral clustering, hierarchical clustering, gaussian mixture models, and combinations thereof.

Many of the algorithms are neural network-based algorithms. A neural network has an input layer, processing layers and an output layer. The information in a neural network is distributed throughout the processing layers. The processing layers are made up of nodes that simulate the neurons by the interconnection to their nodes. Similar to statistical analysis revealing underlying patterns in a collection of data, neural networks locate consistent patterns in a collection of data, based on predetermined criteria.

In other embodiments, principal component analysis is used. Principal component analysis (PCA) involves a mathematical technique that transforms a number of correlated variables into a smaller number of uncorrelated variables. The smaller number of uncorrelated variables is known as principal components. The first principal component or eigenvector accounts for as much of the variability in the data as possible, and each succeeding component accounts for as much of the remaining variability as possible. The main objective of PCA is to reduce the dimensionality of the data set and to identify new underlying variables.

In another embodiment, the algorithm is a classifier. One type of classifier is created by “training” the algorithm with data from the training set and whose performance is evaluated with the test set data. Examples of classifiers are discriminant analysis, decision tree analysis, receiver operator curves or split and score analysis.

In some embodiments, the algorithm or analyzer compares the levels of biomarker combinations of the invention (e.g. total-Tau, p-Tau, Aβ42, and Neuroligin in methods of diagnosing MCI; LC3, TDP43, and NRF2 in methods of diagnosing ALS; or Aβ42, p-Tau, PSD95 and proBDNF in methods of diagnosing AD) as determined in the isolated EV to their respective levels corresponding to a control biofluid sample, to thereby compare the diagnostic signature of the sample to the control diagnostic signature.

Articles of Manufacture

In another aspect there is provided a system for isolating neuron-derived EV, comprising means for identifying or capturing neuron-derived EV from a biofluid sample, comprising surface-bound affinity molecules capable of binding to one or more target molecules on the surface of the EV, and further comprising (i) means for determining the accuracy of the system, and/or (ii) means for determining the levels of at least one biomarker in the captured EV,
wherein at least one target molecule is a synaptic protein,
wherein the means for determining the accuracy of the system comprise particles that display the at least one synaptic protein and are labeled by a marker, and
wherein the at least one biomarker is selected from the group consisting of Tau, p-Tau, Aβ42, NLGN, TDP43, clusterin, SYP, BIM, NEFL, ENO2, NRGN, and GPR26 gene products, and combinations thereof.

In various other embodiments, the biomarkers are selected from the group consisting of the one or more biomarkers are selected from the group consisting of Tau, p-Tau, Aβ42, and NLGN, TDP43, CLU, SYP, BIM, NEFL, ENO2, NRGN, Cathepsin D, LC3, SYT and GPR26 gene products, and combinations thereof. In yet other embodiments, the at least one biomarker is selected from the group consisting of LC3, Cathepsin D, NRF2, Aβ42, p-Tau, PSD95, proBDNF, COX2, EIF2C2 and NF-κB and combinations thereof. Each possibility represents a separate embodiment of the invention.

In another aspect there is provided a kit for identifying or capturing neuron-derived EV, comprising NLGN3-specific affinity molecules and GAP43-specific affinity molecules, capable of binding to NLGN3 and GAP43, respectively, on the surface of EV, and means for identifying or capturing the EV bound to the affinity molecules.

In yet another aspect, there is provided a kit for analyzing a biofluid sample of a subject suspected of having MCI, or of being predisposed to developing AD, comprising means for determining the levels of biomarkers in neuron-derived EV of the sample, wherein the biomarkers are total-Tau, p-Tau, Aβ42, and NLGN3.

In another aspect, there is provided a kit for analyzing a biofluid sample of a subject suspected of having a neurological disorder, comprising means for determining the levels of biomarkers in neuron-derived EV of the sample, wherein:
the subject is suspected of having MCI, or of being predisposed to developing AD, comprising means for determining the levels of biomarkers in neuron-derived EV of the sample, wherein and the biomarkers are total-Tau, p-Tau, Aβ42, and Neuroligin-1;
the subject is suspected of having AD, and the biomarkers are Aβ42, p-Tau, PSD95 and proBDNF; or
the subject is suspected of having ALS, and the biomarkers are LC3, TDP43, and NRF2.

In some embodiments, means for determining the accuracy of the system include in particular, control EV or non-EV particles, as detailed in section C. herein. For example, the system or kit may contain labeled particles that display one or more of the target molecules and/or means for their synthesis, as explained in detail in section C.

In other embodiments, means for determining the levels of at least one biomarker in the captured EV include for example, antibodies, detection probes and the like, amenable for use with any one of the biomarker detection and quantification methods as detailed in section B. herein. It is to be understood, that the means are intended to provide specific identification and/or quantification of the respective biomarker (e.g. antibodies specific to LC3, TDP43, and NRF2 in the case of ALS).

In other embodiments, means for identifying or capturing the EV bound to the affinity molecules include in particular substance-bound affinity molecules directed to specific target molecules as disclosed in section A. herein.

The following examples are presented in order to more fully illustrate some embodiments of the invention. They should, in no way be construed, however, as limiting the broad scope of the invention.

### Examples

Throughout the Examples and as disclosed herein, unless indicated otherwise, the terms “exosomes” and “EV” are used interchangeably.

### Example 1. Screening for Neuronal Selection Targets that Enable Capturing of Neuron-Derived Exosomes (NDE)

20 candidate membrane proteins suggested by bioinformatic analysis were screened for their ability to serve as surface targets for NDE isolation. To this end, 30 different antibodies against these membrane proteins were tested by intact exosome ELISA, as follows. Antibodies were attached to an ELISA plate by overnight incubation, and blocked with BSA and ethanolamine. Plasma samples were added (3 to 243-fold curve serial dilution) and incubated with the plates for two hours. Biotinylated antibodies against the general exosome markers CD63 and CD81 were used in combination with Streptavidin-HRP for detection of the bound EV. The ability of each plate-bound antibody to capture NDE in a specific manner was evaluated by three parameters as presented in Table 3, namely: 1) the signal linearity with dilution evaluated by Pearson correlation (“linearity”), 2) the signal-to-background ratio evaluated by the highest dilution with detectable signal over the blank (no plasma) background (“minimal dilution”) and 3) the ratio between the average of the signal in the top three dilutions and the blank (“fold from blank”). The results are presented in Table 3.

As can be seen in Table 3, several protein markers, including four synaptic proteins and two non-synaptic proteins, produced high quality measurements defined as linearity over 0.9, and signal to background ratio above 10 folds. Notably, neuroligin-3 (NLGN3) and growth-associated protein 43 (GAP43) were found to be significantly more effective than the known target L1 Cell Adhesion Molecule (L1CAM) as surface targets for capturing EV by intact exosome ELISA. These and other high performing candidates were selected for further testing and analysis of their ability to isolate NDE in a selective manner, as described below.

TABLE 3
antibodies tested for NDE isolation

| | | | | |
|---|---|---|---|---|
| Protein | | | Minimal | fold from |
| target | antibody | linearity | dilution | blank |

Synaptic markers

| | | | | |
|---|---|---|---|---|
| NRGN | antibody 1 | 0.494 | 27 | 5.512 |
| | antibody 2 | 0.590 | 81 | 11.133 |
| NLGN1 | antibody 1 | 0.937 | 9 | 4.238 |
| | antibody 2 | 0.895 | 9 | 3.721 |
| NLGN3 | antibody 1 | 0.989 | 243 | 26.773 |
| | antibody 2 | 0.991 | 81 | 23.685 |
| GAP43 | antibody 1 | 0.966 | 243 | 21.216 |
| | antibody 2 | 0.759 | 27 | 6.299 |
| | antibody 3 | 0.809 | 3 | 2.882 |
| SYT | antibody 1 | 0.962 | 27 | 12.628 |
| | antibody 2 | 0.716 | 27 | 7.682 |
| GRIN2B | antibody 1 | 0.836 | 27 | 12.530 |
| GRIA1 | antibody 1 | 0.889 | 27 | 14.930 |
| GRIA2 | antibody 1 | 0.724 | 9 | 11.846 |
| GRIN3a | antibody 1 | 0.865 | 9 | 14.935 |
| SYP | antibody 1 | 0.750 | 81 | 12.659 |
| SYN1 | antibody 1 | 0.443 | 0 | 1.809 |
| | antibody 2 | 0.424 | 3 | 4.320 |
| NLGN4x | antibody 1 | 0.196 | 0 | 1.831 |
| | antibody 2 | 0.382 | 3 | 3.062 |

non-synaptic markers

| | | | | |
|---|---|---|---|---|
| L1CAM | antibody 1 | 0.549 | 3 | 3.893 |
| GPR101 | antibody 1 | 0.955 | 81 | 21.875 |
| GPR26 | antibody 1 | 0.985 | 81 | 25.386 |
| NCAM | antibody 1 | 0.378 | 0 | 1.792 |
| Rab3a | antibody 1 | 0.661 | 27 | 12.203 |
| NRCAM | antibody 1 | 0.500 | 9 | 5.787 |
| | antibody 2 | 0.569 | 0 | 1.342 |
| Ptprz | antibody 1 | 0.619 | 3 | 3.853 |
| | antibody 2 | 0.728 | 0 | 2.127 |
| CNTN2 | antibody 1 | 0.844 | 81 | 17.901 |

### Example 2. Isolation of NDE with Different Selection Targets and Measurement of Intracellular Neural Protein Markers

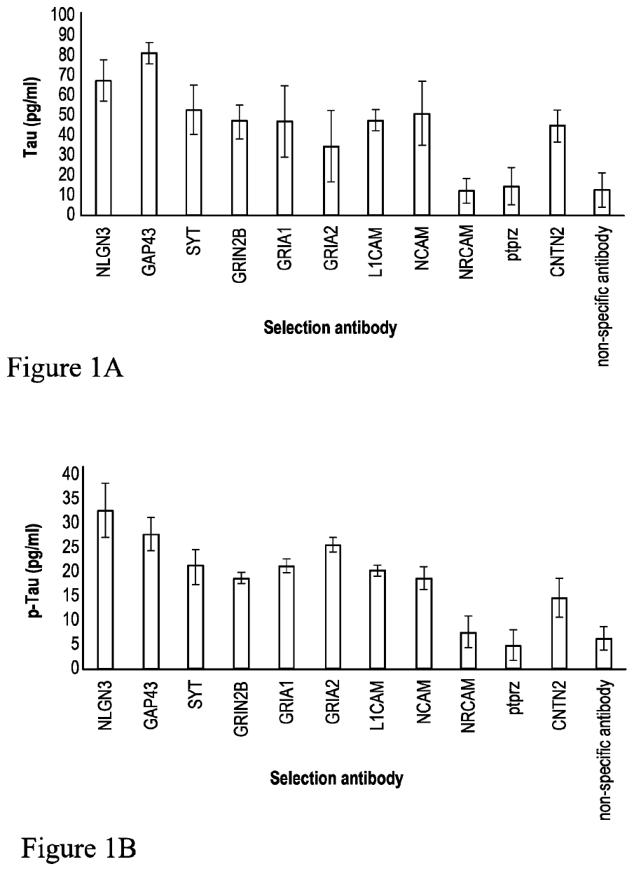

Six synaptic protein markers (NLGN3, GAP43, SYT, GRIN2B, GRIA1 and GRIA2) and five other neuronal protein markers (L1CAM, NCAM, NRCAM, PTPRZ and CNTN2) were tested for their compatibility with NDE isolation from plasma samples using a magnetic bead system, and measurement of intracellular neural protein markers Tau and p-181 phosphorylated Tau (p-Tau). To this end, biotinylated antibodies to the various test markers were attached to streptavidin-conjugated magnetic beads (BioMag® Streptavidin—QIAGEN) by biotin-streptavidin interactions and were added to plasma samples at a concentration of 10 μg/ml and incubated overnight. Following incubation, beads were placed on the magnet rack, EV were washed, eluted and lysed using a detergent-containing buffer (M-PER protein extraction reagent) and the level of Tau and p-Tau were measured using a Luminex multiplex kit according to the manufacturer's instructions. A nonspecific IgG control antibody bound to the magnetic beads was used as a negative control. The results are presented in FIG. 1, in which an average and standard deviation of three independent healthy plasma pools are presented for each antibody.

The results show that both Tau and p-Tau were detectable in the isolated NDE, as opposed to their levels in neat plasma (which were below the assay's lower limit for quantification), and on average higher in EV isolated using synaptic proteins than in EV isolated using non-synaptic proteins as isolation targets. As can be seen in FIG. 1, the levels of both Tau and p-Tau in exosomes isolated with either NLGN3 or GAP43 were about 5-fold higher than in exosomes isolated with non-specific antibody control, whereas their levels in exosomes isolated with L1CAM were only 3-fold higher than control. Thus, NLGN3 and GAP43 were found to be unexpectedly superior to L1CAM in their ability to isolate NDE in a specific manner, manifested by enrichment of neural proteins in the lysate of the isolated EV.

### Example 3. Isolation of NDE with Different Selection Targets and Measurement of Intracellular Neural mRNA Markers

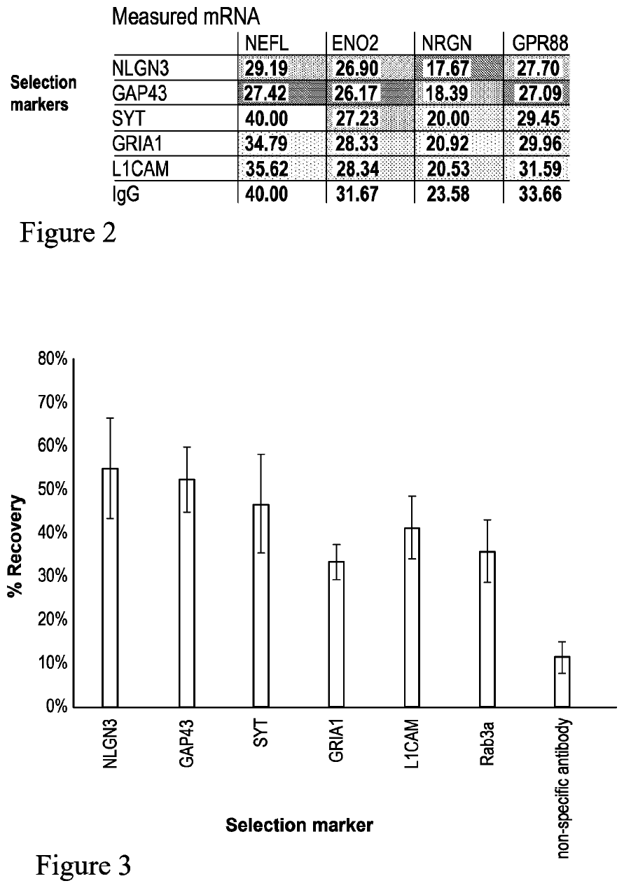

The ability of antibodies against various synaptic proteins (NLGN3, GAP43, SYT or GRIA1) and L1CAM to capture NDE from plasma samples of healthy individuals was compared. The antibodies were attached to magnetic beads, incubated with the plasma samples and then the exosomes were eluted, essentially as described in Example 2. The captured EV were lysed with TRIzol buffer, RNA was isolated from the lysate and corresponding cDNA was made by RT-PCR (thermo fisher 11754050). Preamplification (thermo fisher 4488593) (14ct) and qPCR were performed for several neuron-specific mRNA markers (NEFL, ENO2, NRGN, GPR88) (TaqMan assays: Hs00196245_m1, Hs00157360_m1, Hs00183469_m1 and Hs03027832_s1 respectively). The results are shown in FIG. 2, in which the values are presented as qPCR cycle threshold (Ct) averages of two independent healthy plasma pools (and thus lower number represents higher levels on a log 2 scale); “selection markers” indicate the isolation target to which the antibodies were directed; “IgG” indicates a non-specific antibody control; and “measured mRNA” represents the levels (Ct) of the indicated mRNA markers. Darker shades represent lower Ct, indicative of higher mRNA levels.

As can be seen in FIG. 2, the levels of certain mRNA markers were enhanced in EV isolated with antibodies to the different surface targets. Notably, NLGN3 and GAP43 appeared to be the most selective targets providing enrichment for NDE; the levels of all tested neuronal mRNA were significantly and consistently higher in EV isolated with either NLGN3 or GAP43 than in control non-specific EV. Remarkably, the levels of the various neuronal mRNA were about 8-fold higher in exosomes isolated with NLGN3- or GAP43-specific antibodies than in exosomes isolated with L1CAM-specific antibodies, and at least 64-fold higher than in exosomes isolated with the non-specific antibody control (IgG).

### Example 4. Specific Recovery of Exosomes, Isolated from Human Induced Pluripotent Stem Cells (IPS)-Derived Neurons, Spiked into Plasma Samples

IPS of healthy human donors (purchased from BrainXell) were differentiated ex vivo to cortical neurons, according to the manufacturer's instructions. Conditioned media from the differentiated cortical neurons were collected by a protocol that included collection of 70% of the condition media, twice a week for 4 weeks, centrifugation on 2000 g for 15 min to remove cell debris, and transferring the supernatant to a new tube before freezing. Following thawing of the conditioned media, all collected media were combined into a 200 ml sample, and exosomes where isolated by anion exchange chromatography (Q Column), followed by ultrafiltration on 100 KD cutoff filter, size exclusion chromatography, and finally concentrating the exosomes on a second ultrafiltration 100 KD cutoff filter.

A predetermined amount of the resulting isolated control EV (corresponding to 2000 pg Tau, as determined in a separate assay) was added to 300 μl plasma samples. The plasma samples supplemented (spiked-in) with the predetermined amount of EV were then subjected to the isolation protocol, essentially as described in Example 2, using magnetic beads conjugated to antibodies against the selected surface targets as described in FIG. 3. Magnetic brads conjugated to a non-specific antibody (IgG) served as a control.

The level of Tau was determined by Luminex, and EV recovery was calculated (in %) according to the following formula: (recovered Tau—endogenous Tau)/spiked-in Tau (namely the amount of Tau in the isolated EV, less its level in a native sample prior to the spike-in (which is at least 10 time lower than following the spike-in), divided by the predetermined amount of 2000 pg, present in the spiked-in EV prior to isolation). The results are presented in FIG. 3, each histogram representing an average of three independent healthy plasma pools.

As can be seen in FIG. 3, the recovery rate of the different test antibodies ranged from about 35% for GRIA1 to over 50% for NLGN3 and GAP43, compared to about 10% for the non-specific antibody control; about 40% of the spiked-in EV were recovered with L1CAM-specific antibodies. The fivefold increase in NDE recovery with antibodies to NLGN3 or GAP43 demonstrates their ability to capture NDE in the plasma environment in a specific and efficient manner. Further, the results demonstrate the ability of the system to evaluate the accuracy of the assay by quantifying the proportion of spiked-in EV captured by the system.

### Example 5. System Providing Spike-In Validation Control Using Engineered EV

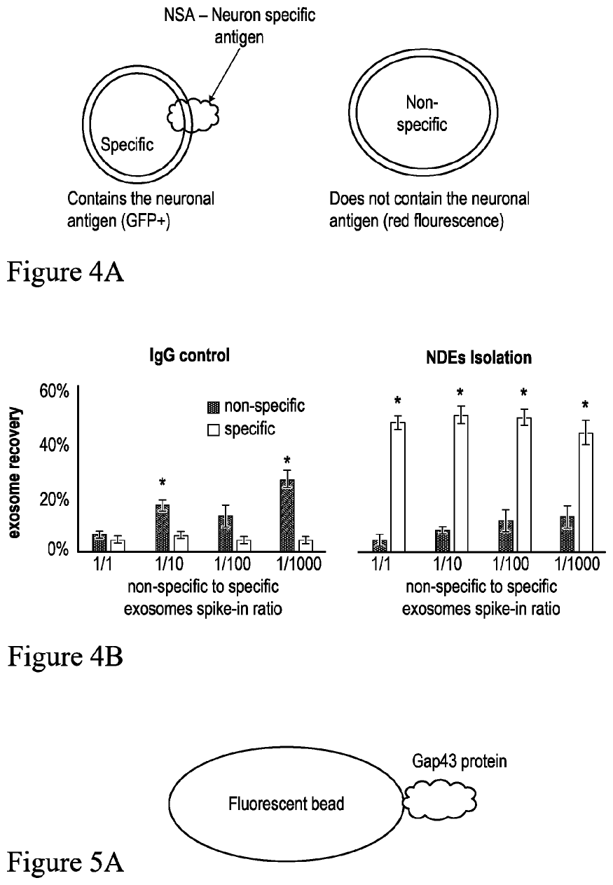

Non-neuronal HEK293 cells were transfected with a recombinant construct (purchased from Sino Biologicals, Cat. No. HG11160-ACG) encoding for NLGN3 and green fluorescent protein (GFP), and a stable genetically-engineered cell line comprising the construct was generated. GFP⁺ EV, containing the green fluorescent marker and the neuron-specific antigen NLGN3 were isolated from conditioned media of these cells by size exclusion chromatography (“specific”, green fluorescent labeled EV). Non-specific EV, obtained from conditioned media of non-transfected HEK293 cells, were labeled with the red fluorescent marker PKH26 (Sigma-Aldrich), according to the manufacturer's instructions (“non-specific”, red fluorescent labeled EV). FIG. 4A provides a schematic illustration of the system, in which “NSA” indicates the neuron-specific target (in this case, NLGN3), used for capturing or identifying the specific EV.

Specific and non-specific labeled EV were spiked into plasma samples at different ratios, as detailed in FIG. 4B. EV were captured from the samples with magnetic beads bound to anti-NLGN3 antibodies or to isotype control antibodies (IgG), essentially as described in Example 2. The recovery rate of specific and non-specific EV was measured in a fluorescent plate reader, and calculated as described in Example 4. The results are presented in FIG. 4B (left panel—IgG control; right panel—NDE isolation using an anti-NLGN3 antibody; * indicates statistical significance between the specific and non-specific recovery rates).

As can be seen in FIG. 4B, the system provided a consistent and reliable measurement of the specific EV recovery, clearly differentiating between the performance of the anti-NLGN3 antibody and the control antibody. Remarkably, a 44-50% recovery rate was consistently evident in the NLGN3-isolated EV, even when the non-specific EV were spiked-in at a 1000-fold excess over the specific EV. The ability to evaluate the reaction accuracy using direct fluorescence measurements provides an improved means for system validation.

### Example 6. Bead-Based System for Internal Spike-In Control

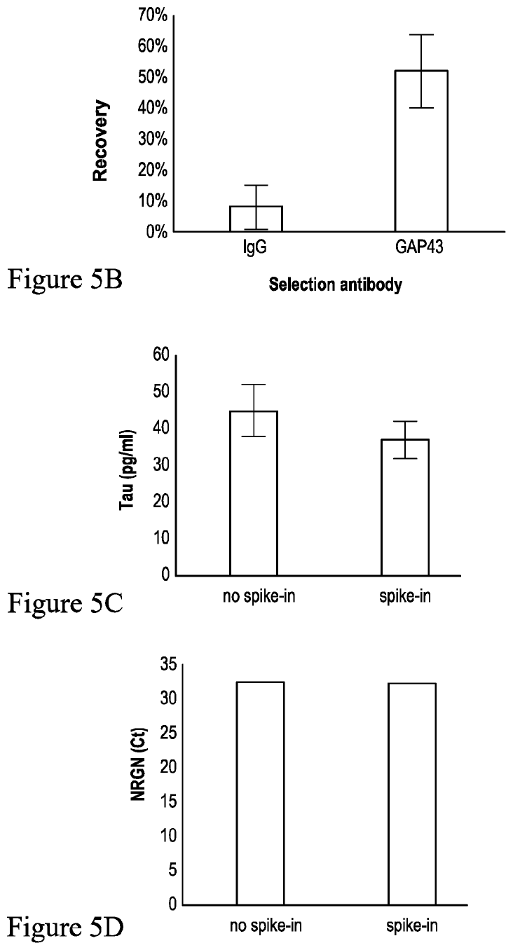

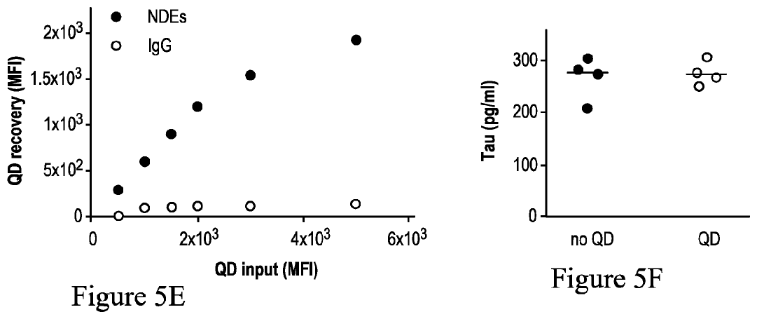

Red fluorescent streptavidin-conjugated beads (Spherotech) were coated with a recombinant biotinylated GAP43 protein by streptavidin-biotin interaction. The beads were spiked into plasma samples at different quantities (fluorescent units), and the minimum amount generating a robust signal was determined and selected for further use. The antigen-presenting labeled beads were captured in the magnetic bead system essentially as described in Example 2, using antibodies specific to GAP43 or non-specific IgG control antibodies (“selection antibodies”). A schematic representation of the labeled beads is provided in FIG. 5A. The recovery rate was evaluated by a fluorescence reader, and the results are presented in FIG. 5B.

As can be seen in FIG. 5B, the recovery of the beads captured using the anti-GAP43 antibody was about 54% (5-fold higher than using the isotype control IgG antibody). Significantly, the measured recovery was found to be highly consistent between different measurements (coefficient of variation (CV)<13%).

Next, the ability of the target-specific labeled beads to serve as an internal spike-in control (that may be added to EV-containing samples), and their potential interference with downstream measurements of various markers in EV isolated from the samples, were evaluated. A representative neuron-specific protein marker (Tau) and a representative neuron-specific mRNA marker (NRGN), were selected for the analysis. To this end, plasma samples, either supplemented with the labeled control beads (“spike-in”) or left untreated (“no spike-in”), were incubated with magnetic beads bound to GAP43-specific antibodies or IgG control antibodies. EV were isolated essentially as described in Example 2, and the levels of the markers in the EV were evaluated, as described in Example 2 (for the protein marker Tau) or Example 3 (for the mRNA marker NRGN). The results are presented in FIG. 5C (Tau) and FIG. 5D (NRGN), respectively.

As can be seen in FIG. 5C-5D, minor, non-significant differences were observed in the levels of the markers measured in EV captured from samples containing the spiked-in beads compared to non-spiked-in samples.

Next, the experiments were repeated with an additional type of labeled particles, namely Quantum dots (QD) nanoparticles. To this end, QD (Thermo Fisher Q10141MP) were coated with a recombinant biotinylated GAP43 protein by streptavidin-biotin interaction, and the labeled, target-specific QD were spiked into plasma samples. The spiked samples were then subjected to EV isolation using a GAP43 antibody (NDE) or a non-specific control antibody (IgG). FIG. 5E plots a recovery curve of the quantum dot particles (QD), showing the rate of mean fluorescent intensity (MFI) measured following isolation (“QD recovery”) as a function of the initial amount of spiked-in QD (“QD input”).

FIG. 5F demonstrates that QD spike-in does not interfere with the measured level of Tau in the NDE isolated from the spiked-in sample.

Thus, the results demonstrate the compatibility of the system for use as an internal spike-in control, providing for evaluating the recovery rate of each test sample without significantly affecting the yield of the assay. The results demonstrate that both microparticles and nanoparticles are amenable for use as an internal control.

### Example 7. Synergistic Combinations of Antibodies to Multiple Isolation Targets Provide Highly Selective NDE Isolation

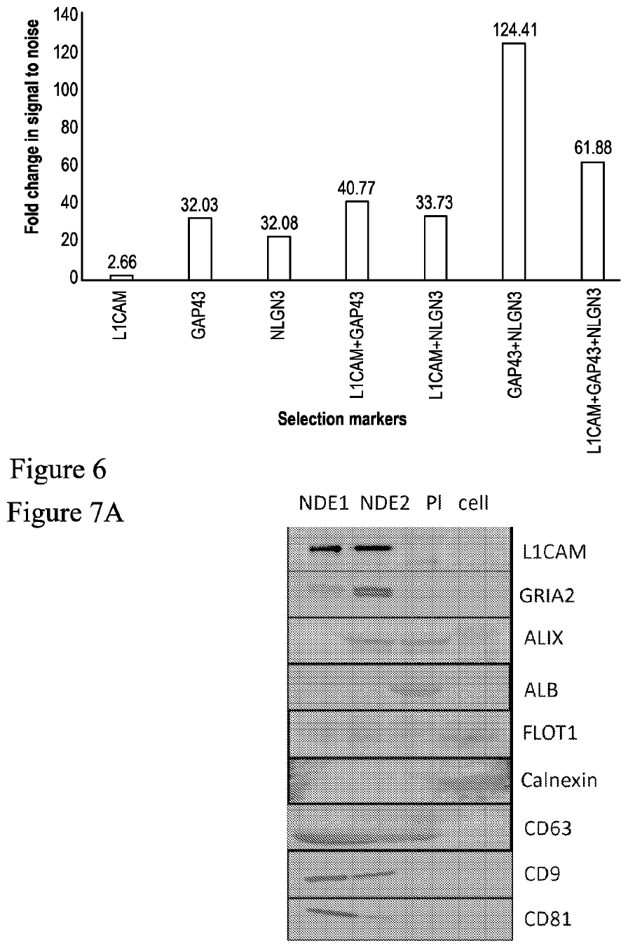

Antibodies against L1CAM, NLGN3 and GAP43 (“selection markers”) were tested, either alone or in various combinations, for their ability to isolate NDE from plasma samples. To this end, antibodies were conjugated to magnetic beads and used to capture EV essentially as described in Example 2. The levels of the neuronal mRNA marker NRGN (signal) and the platelet mRNA marker PF4 (noise) were measured in each sample lysate, essentially as described in Example 3. The signal to noise (NRGN/PF4) ratio in the EV isolated with each antibody or combination was compared to the ratio in total plasma RNA (isolated with Qiagen RNAeasy kit). The results are presented in FIG. 6.

As can be seen in FIG. 6, both NLGN3 and GAP43 were about 10-fold more effective than L1CAM as selective NDE isolation targets. Further, antibodies to NLGN3 or GAP43 used in combination with anti-L1CAM antibodies, enhanced this ability in a greater than additive or synergistic manner, improving the signal-to-noise gain to 40.77 folds (L1CAM+GAP43, compared to 2.66 for L1CAM alone and 32.03 folds for GAP43 alone) or 33.73 folds (L1CAM+NLGN3, compared to 2.66 for L1CAM alone and 23.08 folds for NLGN3 alone). Combining the three antibodies improved the selective capture ability in a synergistic manner, enhancing the signal-to-noise gain to 61.88 folds. Even more remarkably, the synergism was greater when the beads contained antibodies to NLGN3 and GAP43, without the addition of antibodies to L1CAM. The signal-to noise gain was more than double than with beads conjugated to all three antibodies, and 4-5 folds higher than with each antibody alone; notably the signal-to noise gain for the combination of NLGN3- and GAP43-specific antibodies (124.41) compared to L1CAM-specific antibodies (2.66) was of about 47-folds (FIG. 6).

The results demonstrate the advantageous use of a combination of antibodies to synaptic proteins, either alone or further targeting L1CAM, in selectively capturing NDE from biological samples. The results further demonstrate that the combined use of NLGN3 and GAP43 as isolation targets provides a highly selective and synergistic enrichment of specific neural markers, compared to non-specific platelet markers, in the isolated EV.

### Example 8. Evaluation of the Specificity of the Isolation Process Using Western Blot and FACS Analyses

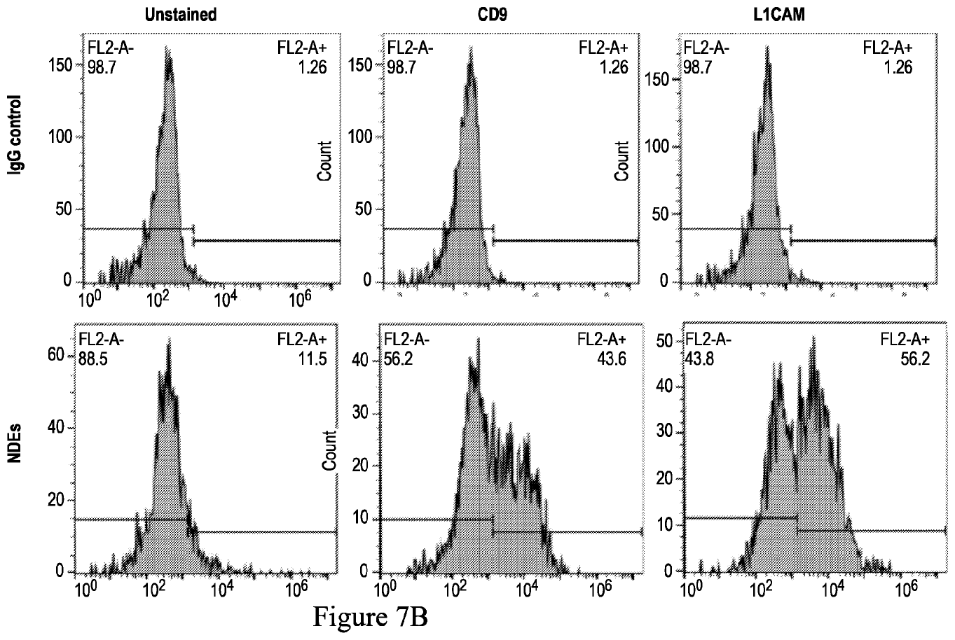

In order to further verify that the particles isolated by the antibody-bound magnetic beads are NDE, plasma samples of two donors (“NDE1” and “NDE2”) were subjected to the isolation protocol essentially as described in Example 7, using a combination of L1CAM, NLGN3 and GAP43-specific antibodies. Total Plasma (P1) and HEK293 cell lysate (cell) that were not subjected to the isolation protocol were used as controls. The levels of the neuronal proteins L1CAM and GRIA2, as well as the general exosome markers ALIX, FLOT1, CD63, CD9 and CD81, were determined in the lysates of the captured EV or the control samples using Western blotting (WB), using standard procedure. The levels of albumin (“ALB”), a common non-exosomal plasma protein, and of calnexin, a cellular protein not released in exosomes, were used as negative controls. The results are shown in FIG. 7A.

In addition, EV were captured from plasma samples with magnetic beads coated with NLGN3, GAP43 and L1CAM-specific antibodies, or with a non-selective IgG control, essentially as described in Example 7, with the exception that instead of using the elute and lysing the eluted EV, a second antibody against L1CAM or CD9, conjugated to the fluorophore (R-phycoerythrin) was used to interact with and detect the bound EV. Fluorescence was measured by fluorescence-activated cell sorting (FACS), and the results are shown in FIG. 7B.

The results demonstrate the specific isolation of NDE using both WB and FACS. The very low levels of albumin and calnexin detected in the captured EV lysates indicate significant purity of the isolation process with minimal contamination of unrelated markers.

This was further confirmed by an additional automated Western blot assay (WES™ instrument manufactured by ProteinSimple), in which the purity and specificity of NDE isolated as described in Example 7 were examined using antibodies against the exosomal markers CD9 and FLOT1, as well as against the neuronal markers NeurN and GluR2. The additional markers also demonstrated the high purity and specificity of the isolated NDE.

### Example 9. Demonstration of Specific Enrichment for Neuronal Proteins by Mass Spectrometry, and of Compatibility with Proteomic and Lipidomic Analyses

Antibodies against NLGN3, GAP43 and L1CAM were attached to a batch of magnetic beads and used to capture NDE from a pool of plasma samples of healthy human individuals, essentially as described in Example 7. Following incubation with the beads, the captured EV were eluted, lysed and subjected to liquid chromatography tandem mass spectrometry (LC-MS-MS) and proteomic analysis.

620 unique proteins were identified on average, namely a number within the range typically identified in proteomic analyses of untreated plasma samples. Gene ontology bioinformatic analysis (using the database for annotation, visualization and integrated discovery bioinformatics resources—DAVID) was performed. The results are presented in Table 4, in which the ten most significant tissue “go-term” identifiers (“PV” represent P-values of the gene ontology analysis, “2.00E-138” represents 2×10⁻¹³⁸, etc.) for each group (for total exosomes and NDE) are presented.

TABLE 4
proteomic analysis

| | Total EV | NDE |
|---|---|---|
| Plasma | 2.00E−138 | |
| Liver | 3.40E−57 | 8.00E−06 |
| Lung | 2.60E−05 | 5.10E−02 |
| Lymphocyte | 8.80E−14 | 2.50E−01 |
| Skin | 9.20E−03 | |
| Corpus callosum | 9.50E−08 | 8.40E−03 |
| Cerebrospinal fluid | 8.30E−02 | 8.00E−03 |
| Brain cortex | | 3.50E−05 |
| Substantia nigra | | 6.80E−02 |
| Cajal-Retzius cell | 6.00E−18 | 6.30E−59 |
| Fetal brain cortex | 2.90E−17 | 3.30E−57 |
| Brain | | 2.50E−06 |
| Fetal brain | | 1.90E−03 |
| Amygdala | | 1.50E−02 |
| Hippocampus | | 1.00E−01 |

As can be seen in Table 4, the analysis revealed a significant enrichment for brain-related proteins in EV isolated with the specific antibodies (“NDE”) over total EV, isolated by the ExoQuick Kit. The list of enriched proteins identified exclusively or preferentially in NDE contained several neuron-specific proteins.

The lipid composition of the isolated NDE, total EV, and EV obtained using a non-selective IgG control, was further analyzed using mass spectrometry specific to lipid analysis. The results are presented in Table 5, in which, for each lipid marker, its percentile of the total amount of lipids identified in each sample, is presented.

As can be seen in Table 5, the identified lipids were characteristic of exosomes, and the proportions of lipids found in the isolated NDE were significantly different from those found in total exosome or the control sample (obtained using non-selective IgG).

TABLE 5
lipidomic analysis

| Lipid | IgG | NDE | Total EV |
|---|---|---|---|
| FC | 7.0% | 10.5% | 30.4% |
| CE | 67.8% | 57.5% | 50.4% |
| AC | 0.0% | 0.2% | 0.0% |
| MG | 0.1% | 1.0% | 0.0% |
| DG | 0.0% | 0.0% | 0.1% |
| TG | 6.4% | 2.8% | 2.1% |
| Cer | 0.1% | 0.1% | 0.1% |
| dhCer | 0.0% | 0.0% | 0.0% |
| SM | 4.7% | 3.0% | 5.1% |
| dhSM | 0.1% | 0.1% | 0.2% |
| MhCer | 0.1% | 0.1% | 0.1% |
| Sulf | 0.0% | 0.0% | 0.0% |
| LacCer | 0.0% | 0.0% | 0.2% |
| GM3 | 0.0% | 0.0% | 0.1% |
| GB3 | 0.0% | 0.0% | 0.0% |
| PA | 0.0% | 0.0% | 0.0% |
| PC | 12.3% | 18.7% | 7.0% |
| PCe | 1.0% | 1.9% | 1.3% |
| PE | 0.0% | 0.1% | 0.5% |
| PEp | 0.1% | 0.7% | 0.8% |
| PS | 0.0% | 0.6% | 0.0% |
| PI | 0.2% | 0.4% | 0.9% |
| PG | 0.0% | 0.0% | 0.0% |
| BMP | 0.0% | 0.0% | 0.0% |
| AcylPG | 0.0% | 0.0% | 0.0% |
| LPC | 0.0% | 1.7% | 0.6% |
| LPCe | 0.0% | 0.0% | 0.0% |
| LPE | 0.0% | 0.3% | 0.1% |
| LPEp | 0.0% | 0.0% | 0.0% |
| LPI | 0.0% | 0.0% | 0.0% |
| LPS | 0.0% | 0.1% | 0.0% |
| NAPE | 0.0% | 0.0% | 0.0% |
| NAPS | 0.0% | 0.0% | 0.0% |
| NSer | 0.0% | 0.0% | 0.0% |

Thus, the results demonstrate, using an unbiased proteomic approach, that EV isolated with a combination of NLGN3, GAP43 and L1CAM antibodies are significantly enriched for NDE. The results further demonstrate the compatibility of the assay with proteomic and lipidomic analyses.

### Example 10. The Levels of Neural Proteins in NDE are Positively Correlated with Cortex and Hippocampus Levels in Several Alzheimer's Disease (AD) Mouse Models

NDE were isolated from murine plasma samples with antibodies against NLGN3, GAP43 and L1CAM, essentially as described in Example 7. The mice examined were either wild-type mice, or mice strains corresponding to of various models of AD. The following cohorts of mice were examined: n=4 2×Tg-AD (all female; age: mean=8.3, SD=0.12 months; squares), n=15 3×Tg-AD (9 male, 6 female; age: mean=9.1, SD=0.8, 6-10.5 months; crosses), n=9 5×FAD (all female; 12 months old; stars) and n=15 wild type mice (WT; 5 male, 10 female; age: mean=8.9, SD=3.2, 5-12 months; circles); total of 43 mice; 29 female, 14 male; age: mean=9.6, SD=2.4, 5-12 months.

The selected models represent diverse mice models of AD, which have been extensively characterized and accepted as models for AD. In particular, the models represent forms of familial AD with different pathologies. The 2×Tg-AD amyloidosis mouse model expresses the human amyloid precursor protein (APP) KM670/671NL (Swedish; APP_{swe}) and presenilin-1 (PSEN1) DE9 mutations in the central nervous system (CNS) under the control of the mouse prion protein promoter, resulting in pathological APP processing and accumulation of Aβ plaques. The 5×FAD is a newer model that recapitulates Aβ pathology more rapidly by overexpressing human APP and PSEN1 transgenes with a total of five mutations (APP_{swe}, APP I716V (Florida), APP V717I (London), PSEN1 M146L (A>C), and PSEN1 L286V). 3×Tg-AD mice exhibit both Aβ plaque and Tau tangle pathologies, by overexpressing human APP_{swe}, and Tau P301L in PSEN1 M146V mutant knockin mice. Astrocytic complement expression is elevated in mouse models of tauopathy and amyloidosis. Aβ striking C1q protein level increase in the P301S mouse model of tauopathy is associated with synaptic degeneration. The different mice represent slightly different aspects and severity of the disease and, thus, allow the evaluation of the concordance between plasma EV and the CNS tissue.

The levels of several neural proteins and control proteins were measured in the isolated EV (“NEVs”), as well as in dissected cortex and hippocampus tissues of each mouse (“cortex”), using a Luminex multiplex assay.

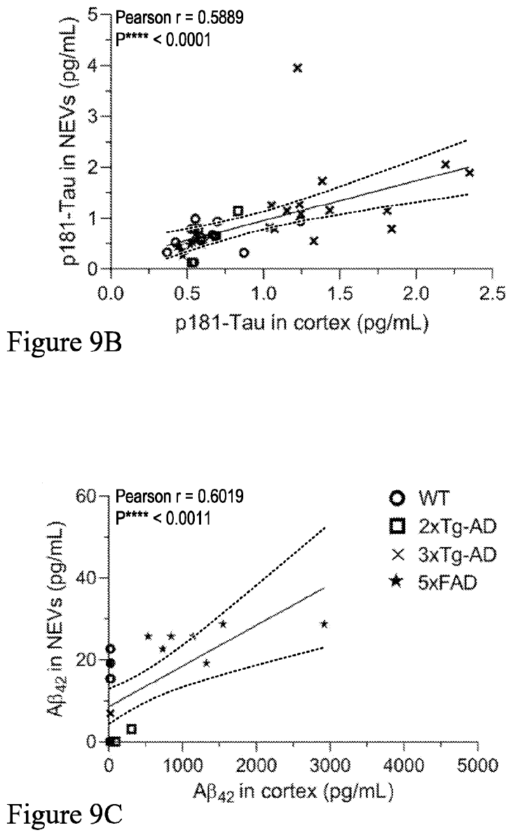

The results show statistically significant positive correlations between the levels of total Tau (tTau), phosphorylated Tau (p181-Tau) and amyloid beta 42 (Aβ42; FIG. 9A-9C, respectively) in NDE and their levels in cortex and hippocampus tissues, as indicated by the Pearson correlation coefficient shown in each graph. Remarkably, the level of these proteins in the isolated NDEs were also correlated with the age of the mice and the severity of their brain pathology. This demonstrates that the exosomes isolated with a combination of NLGN3, GAP43 and L1CAM-specific antibodies reflect changes occurring in the murine brain, and provide a useful tool to probe the brain by a minimally invasive blood test.

Similar results were obtained for the neural proteins clusterin and SYP, as well as for BIM, a non-tissue-selective protein, found in all cell types, which also showed significant correlation between their levels in the isolated NDE and their levels in brain tissue. These results further demonstrate the ability of NDE isolated as described herein to provide a proportional representation of housekeeping proteins, reflecting their relative levels in the tissue of origin.

### Example 11. Development of Highly Accurate Diagnostic Classifiers for Mild Cognitive Impairment (MCI) and Alzheimer's Disease (AD)

A cohort of patients with mild cognitive impairment (MCI) including MCI associated with early AD (Mini-Mental State Examination (MMSE) score=27±3.2, n=20), and matched normal controls (n=20) were analyzed in a blind manner, as follows. NDEs were isolated from plasma samples with antibodies against NLGN3, GAP43 and L1CAM (hereinafter “the combined targets”), essentially as described in Example 10. The levels of various proteins were measured in the NDE lysates using Luminex or ELISA analyses. The efficacy of the different proteins as diagnostic candidates was evaluated bioinformatically, by comparison to their respective levels in samples of healthy control individuals. For evaluating the levels of NLGN1, the MBS9313140 NLGN1 ELISA kit (MyBioSource) was used. This kit is useful for identifying Neuroligin (NLGN) family members and in particular human Neuroligin-1 (NLGN1).

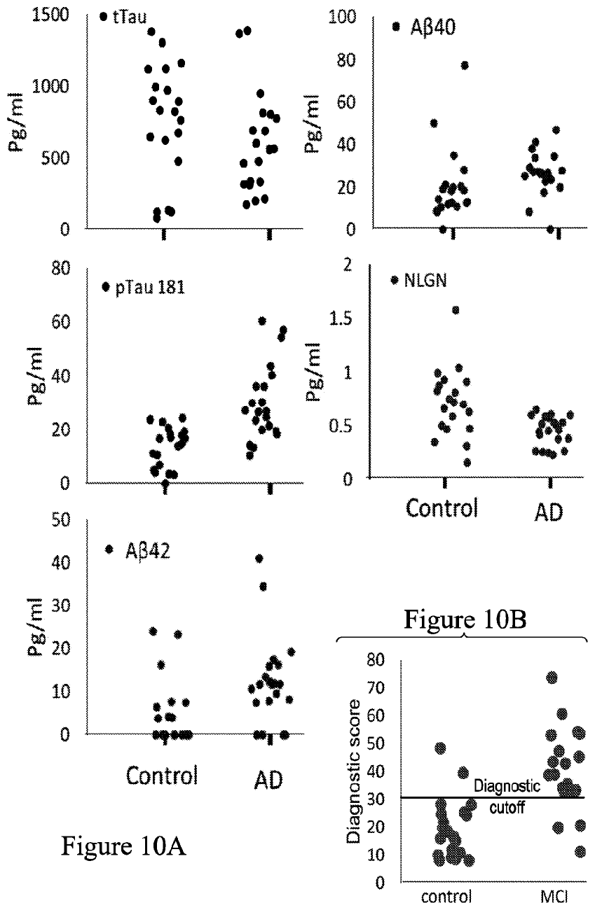

The levels of some of the proteins measured in the individual patients are shown in FIG. 10A. As can be seen in FIG. 10A, while some of these proteins showed, on average, a trend towards enhancement (e.g. pTau, Ab42) or reduction (NLGN1) in MCI patients (right) compared to healthy individuals (left), no individual protein was sufficient to differentiate between the groups with sufficient accuracy, nor to provide a reliable diagnosis for specific patients. Other proteins, such as SYP, cathepsin D, and HSP70, were not effective as diagnostic markers and did not contribute to the separation of the groups.

Surprisingly, a protein signature was identified, providing high accuracy in separating between patients with MCI and healthy controls. Specifically, a diagnostic algorithm based on the combined relative levels of four proteins, namely Tau, p-Tau, Neuroligin and amyloid-beta 42, yielded 85% sensitivity and 90% specificity. The results are shown in FIG. 10B, in which “Diagnostic score” is a numerical score reflecting the combined relative levels of these proteins calculated according to the algorithm. The diagnostic cutoff, namely the threshold for MCI determination found applicable to the analysis of this group of test subjects, is also shown in FIG. 10B.

The aggregation of Tau and amyloid-β (Aβ) isoforms in the brain is characteristic of AD; thus, they are considered as candidate biomarkers. However, research attempting to establish the reliability of Aβ and Tau as biomarkers in blood samples has culminated in an amalgamation of contradictory results and theories regarding the conditions and parameters necessary for an accurate diagnosis. Although various blood biomarkers were hitherto suggested, none has yet been established or implemented in early diagnostic protocols. AD is still considered to be an untreatable and incurable disease because, by the time symptoms appear in patients, the disease has progressed to a point where most therapeutic agents are rendered ineffective.

Without wishing to be bound by a specific theory or mechanism of action, the method described herein, comprising NDE isolation using the combined targets, and payload analysis using the four-marker protein signature as disclosed herein, provided an unexpectedly accurate diagnosis of MCI and early detection of an underlying condition of AD, compared to hitherto suggested blood tests.

In a similar experiment, an additional set of proteins was tested for the ability to assess the difference between AD and healthy controls. Plasma NDE were isolated (using antibodies against NLGN3, GAP43 and L1CAM as described above) from early AD patients (three cohorts of 10, 20 and 32 patients) and healthy control subjects. In contradistinction from the patients in the MCI cohort (in which the MMSE score ranged from 25-30), the patients in the AD cohorts exhibited dementia, and were characterized by a MMSE score ranging from 18-27.

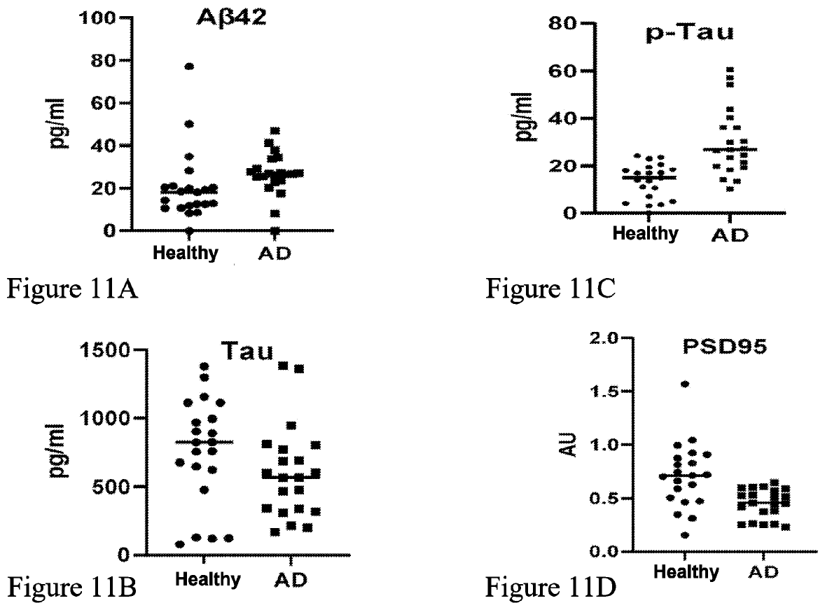

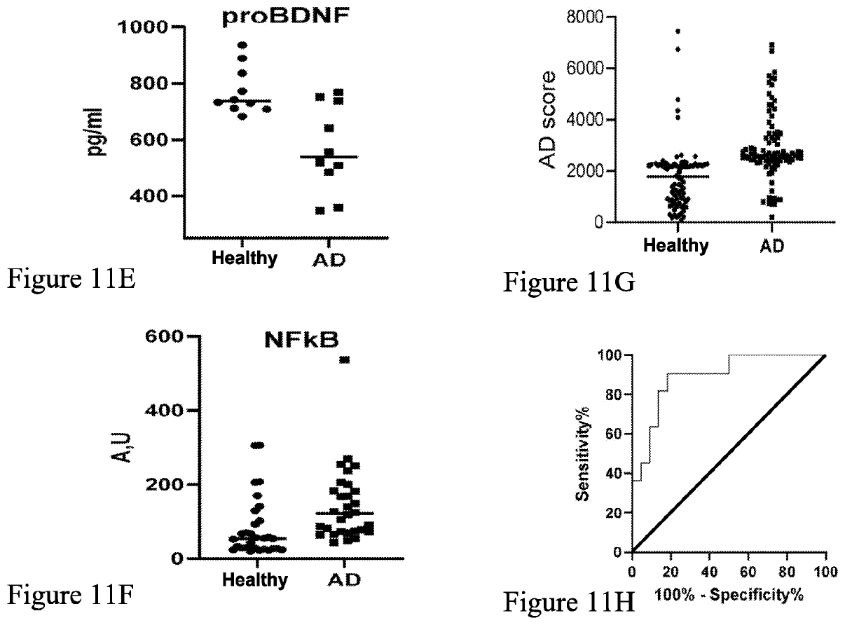

The results are presented in FIG. 11A-11F (in pg/ml (FIGS. 11A, 11B, 11C and 11E, or in relative units (A,U) in FIGS. 11D and 11F). As can be seen in FIG. 11A-11F, the levels of the following proteins were different between AD and healthy controls: Aβ42 (FIG. 11A), total Tau (Tau, FIG. 11B), p-Tau (p-181-Tau, FIG. 11C), PSD95 (FIG. 11D), BDNF precursor (proBDNF, FIG. 11E) and NfkB (FIG. 11F).

Further, as can be seen if FIG. 11G-11H, a diagnostic signature, combining the levels of Aβ42, p-181-Tau, PSD95 and proBDNF, was unexpectedly found to differentiate between the groups with exceptional accuracy.

As presented in FIG. 11G, “AD score” is a numerical score reflecting the combined relative levels of these proteins calculated according to the algorithm (combining the levels Aβ42, p-181-Tau and reducing the levels of PSD95 and proBDNF at predetermined ratios). The diagnostic cutoff, namely the threshold for AD determination found applicable to the analysis of this group of test subjects, is also shown in FIG. 11G.

FIG. 11H shows a Receiver Operator Characteristic (ROC) curve based on combination of Aβ42, p-181-Tau, PSD95 and proBDNF levels for AD patients vs control. Remarkably, an analysis of 62 AD patients and 65 controls revealed that they can be separated with 90% sensitivity and 78% specificity (FIG. 11H).

### Example 12. Differential Diagnosis of Frontotemporal Dementia Pathophysiology

Frontotemporal dementia (FTD) encompasses a spectrum of clinical syndromes associated with brain accumulation of different proteins, including mainly Tau and Transactive response DNA binding protein of 43 kDa (TDP43). Early detection of the underlying pathology would be highly advantageous in the development and determination of treatments. However, to date, the proteinopathies can only be suggested based on the type of symptoms, in a highly error-prone manner; accurate diagnosis can only be done postmortem.

A study was conducted on nine blood samples obtained from patients with sporadic behavioral FTD. Plasma NDEs were isolated by the combined targets essentially as described in Example 7. p181-Tau (p-Tau) and TDP43 levels in the NDE lysates were measured by Luminex and ELISA, respectively, and normalized to the respective average values in all patients.

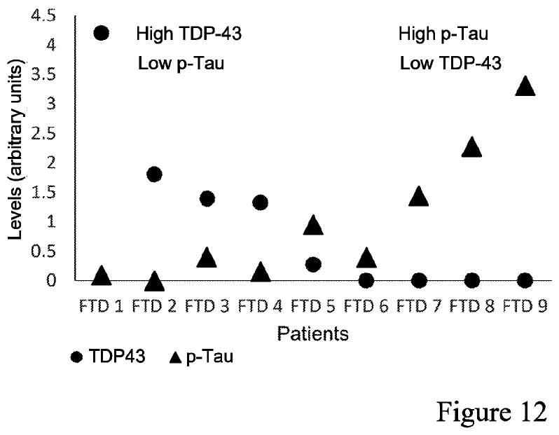

The results are presented in FIG. 12, in which FTD1 to FTD9 represent the individual patients, and the normalized levels of p-Tau (triangles) and TDP43 (circles) are presented in arbitrary units calculated as the level of the marker in EV of the patient divided by the average value in the test population, according to the formula: normalized value=value₍ₚₐₜᵢₑₙₜ₎/value_{(average)}. Further, the levels of the protein markers in NDE isolated from the individual patients (in pg/ml) and the respective ratios of TDP43 levels to p-Tau levels, are shown in Table 6 below.

TABLE 6
levels of biomarkers in EV of FTD patients

| TDP43 | p-Tau | ratio |
|---|---|---|
| 715.429 | 4.256 | 168.107 |
| 307.286 | 3.000 | 102.429 |
| 236.643 | 18.385 | 12.871 |
| 226 | 7.139 | 31.656 |
| 47 | 44.046 | 1.0671 |
| 0 | 154.905 | 0 |
| 0 | 106.693 | 0 |
| 0 | 18.426 | 0 |
| 0 | 67.634 | 0 |

As can be seen in FIG. 12 and Table 6, a combination of the markers TDP43 and p-Tau clearly identified two distinct FTD patient groups, characterized by high NDE TDP43 levels and low NDE p-Tau levels, or vice a versa.

Accordingly, the method disclosed herein, comprising isolation of plasma NDEs by the targets as disclosed herein and payload analysis using TDP43 and p-Tau as diagnostic markers, provide for early detection and determination of FTD pathology. The results presented herein may provide the first blood biomarkers for FTD pathology, having a highly important role in treatment development.

### Example 13. Development of Highly Accurate Diagnostic Classifier for Amyotrophic Lateral Sclerosis (ALS)

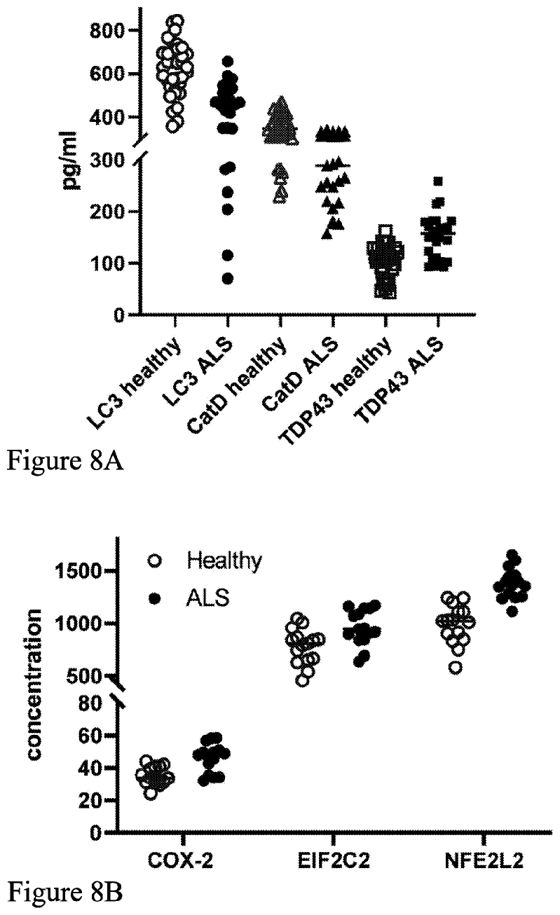

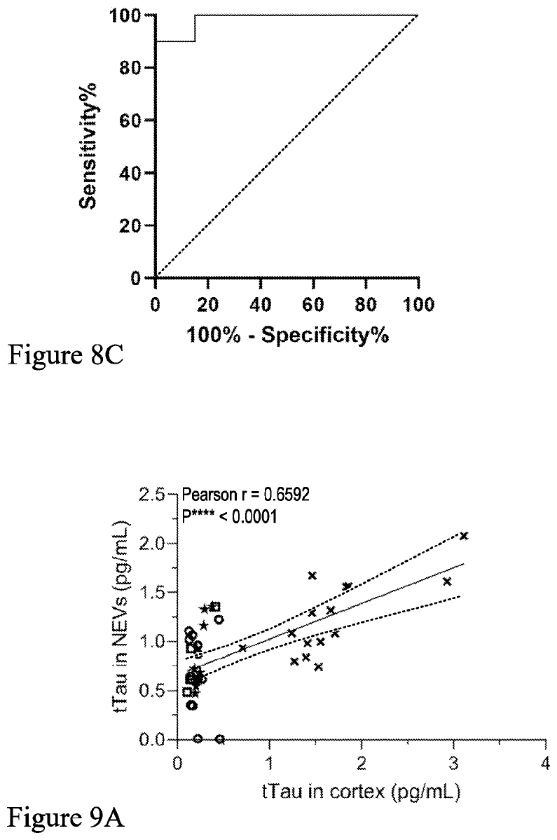

The potential of using NDE protein biomarkers for diagnosing ALS was also examined. A cohort of patients with Amyotrophic lateral sclerosis (ALS, n=35), and matched normal controls (healthy, n=35) were analyzed. NDEs were isolated from plasma samples using antibodies against NLGN3, GAP43 and L1CAM essentially as described in Example 11. The levels of various proteins were measured in the NDE lysates using Luminex or ELISA analyses (LC3 and Cathepsin D R&D system, TDP43 MyBioSource, COX2, EIF2c2 and NFE2L2, CUSABIO). The efficacy of the different proteins as diagnostic candidates was evaluated, by comparison to their respective levels in EV obtained from samples of healthy control individuals. The levels of some of the proteins measured in the individual patients are shown in FIGS. 8A and 8B.

As can be seen in FIGS. 8A and 8B, the levels of certain protein biomarkers, including LC3, Cathepsin D (CatD), TDP43 and NRF2 (NFE2L2), differed between EV obtained from ALS patients and those obtained from healthy individuals. The difference was less pronounced for COX-2 and EIF2C2, and other biomarkers, including proBDNF and BAD, exhibited no substantial difference between the samples. Surprisingly, a protein signature was identified, providing high accuracy in separating between patients with ALS and healthy controls. Remarkably, a diagnostic algorithm based on the combined relative levels of LC3, TDP43, and NRF2 (NRF2+TDP43-LC3 at a predetermined ratio), yielded 90% sensitivity and 100% specificity (FIG. 8C).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-77. (canceled)

78. A method of identifying or capturing neuron-derived EV, comprising:
a. providing an EV-containing biofluid sample,
b. contacting the sample with affinity molecules capable of binding to target molecules on the surface of the EV, under conditions enabling specific binding of the affinity molecules to their corresponding target molecules, wherein the target molecules comprise NLGN3 and GAP43, and
c. identifying or capturing the EV bound to the affinity molecules.

79. The method of claim 78, wherein the target molecules are NLGN3 and GAP43, or wherein the target molecules are NLGN3, GAP43 and L1CAM.

80. The method of claim 78, wherein the affinity molecules are substance-bound, and wherein the substance is a plurality of magnetic beads.

81. The method of claim 80, further comprising:
i. providing a control sample containing a predetermined amount of particles that display at least one of the target molecules,
ii. contacting the control sample with the affinity molecules, under the conditions enabling specific binding to their corresponding target molecules,
iii. quantifying the amount of particles bound by the affinity molecules, and
iv. determining that the amount of bound particles is above a predetermined threshold.

82. The method of claim 81, wherein the particles are fluorescently labeled beads, and the control sample is combined with the biofluid sample prior to contacting the biofluid sample with the affinity molecules.

83. The method of claim 81, wherein the control sample comprises positive control EV engineered to express the one or more of the target molecules exogenously, and optionally further comprises negative control EV that do not express the target molecules, or wherein the control sample comprises negative control EV obtained from non-neuronal cells that contain a first fluorescent marker, and positive control EV obtained from equivalent non-neuronal cells engineered to express the one or more of the target molecules, and containing a second, distinct fluorescent marker.

84. The method of claim 81, wherein the predetermined threshold corresponds to recovery of at least 44% of the predetermined amounts of particles that display the one or more of the target molecules, provided in the control sample.

85. The method of claim 78, wherein the isolated EV are characterized by a ratio of neuron-specific marker levels to non-neuron-specific marker levels of at least 20-fold.

86. The method of claim 78, further comprising determining the levels of one or more biomarkers in the EV bound to the affinity molecules, wherein the biomarkers are selected from the group consisting of protein biomarkers, nucleic acid biomarkers, lipid biomarkers, metabolite biomarkers, and combinations thereof.

87. The method of claim 86, wherein the biomarkers are selected from the group consisting of Tau, phosphorylated Tau (p-Tau), Amyloid-beta 42 (Aβ42), and NLGN, TDP43, clusterin, SYP, BIM, NEFL, ENO2, NRGN, and GPR26 gene products, and combinations thereof.

88. The method of claim 87, wherein the biomarkers are Tau, p-Tau, Aβ42, and Neuroligin, and wherein the biofluid sample is obtained from a subject suspected of having MCI, or of being predisposed to developing AD.

89. The method of claim 87, wherein the sample is obtained from a subject diagnosed with, or suspected of having, frontotemporal dementia (FTD), and the biomarkers are TDP43 and p-Tau.

90. The method of claim 87, wherein the one or more biomarkers are selected from the group consisting of: LC3, Cathepsin D, NRF2, Aβ42, p-Tau, PSD95, proBDNF, COX2, EIF2C2 and NF-κB.

91. The method of claim 90, wherein the biomarkers are Aβ42, p-Tau, PSD95 and proBDNF, and the sample is obtained from a subject suspected of having AD.

92. The method of claim 87, wherein the biomarkers are LC3, TDP43, and NRF2, and the sample is obtained from a subject suspected of having amyotrophic lateral sclerosis (ALS).

93. The method of claim 78, wherein the affinity molecules are antibodies or comprise an antigen-binding portion thereof.

94. The method of claim 78, wherein the biofluid sample is selected from the group consisting of blood, plasma and serum, or wherein the subject is human.

95. A system for isolating neuron-derived extracellular vesicles (EV), comprising means for identifying or capturing neuron-derived EV from a biofluid sample, comprising substance-bound affinity molecules capable of binding to target molecules on the surface of the EV, and further comprising (i) means for determining the accuracy of the system, and/or (ii) means for determining the levels of at least one biomarker in the captured EV,
wherein at least one of the target molecules is a synaptic protein selected from the group consisting of NLGN3 and GAP43,
wherein the means for determining the accuracy of the system comprise particles that display the at least one synaptic protein and are labeled by a marker, and
wherein the at least one biomarker is selected from the group consisting of Tau, phosphorylated Tau (p-Tau), Amyloid-beta 42 (Aβ42), NLGN, TDP43, clusterin, SYP, BIM, NEFL, ENO2, NRGN, and GPR26 gene products, and combinations thereof, or selected from the group consisting of LC3, Cathepsin D, NRF2, Aβ42, p-Tau, PSD95, proBDNF, COX2, EIF2C2 and NF-κB and combinations thereof.

96. The system of claim 95, wherein the target molecules comprise NLGN3 and GAP43, or wherein the target molecules are NLGN3 and GAP43, or wherein the target molecules are NLGN3, GAP43 and L1CAM.

97. A kit for identifying or capturing neuron-derived extracellular vesicles (EV), comprising NLGN3-specific affinity molecules and GAP43-specific affinity molecules, capable of binding to NLGN3 and GAP43, respectively, on the surface of EV, and means for identifying or capturing the EV bound to the affinity molecules.
